(19) United States
(12) Patent Application Publication
(10) Pub. No.: US 2025/0376476 A1
(43) Pub. Date: Dec. 11, 2025

(54) Crystalline forms of a HER2 tyrosine kinase inhibitor

(71) Applicant: Boehringer Ingelheim International GmbH, Ingelheim am Rhein (DE)

(72) Inventors: Jason Alan MULDER, New Milford, CT (US); Ghada AYOUB, Etobicoke (CA); Bahareh KHALILI NAJAFABADI, Oakville (CA); Shanming KUANG, Chester Springs, PA (US); Elnaz LATIFI, Guelph (CA); Vaijinath Dattatrya MANE, Mississauga (CA); Harsh S. SHAH, Monroe, NJ (US); Matthew WATHIER, Oakville (CA)

(21) Appl. No.: 19/233,315

(22) Filed: Jun. 10, 2025

(30) Foreign Application Priority Data
Jun. 11, 2024 (EP) 24181384.9

(51) Int. Cl. C07D 487/04

(52) U.S. Cl. CPC C07D 487/04

(57) ABSTRACT

The present invention relates to crystalline forms of zongertinib, including crystalline forms XXIV, XIX, XVIII, and XX, which are particularly suitable as intermediates in large scale manufacturing of solid state forms of zongertinib and to methods for their preparation. The present invention also includes methods to make zongertinib crystalline forms III, IV, or amorphous using the crystalline forms of zongertinib disclosed herein, as well as solid forms of zongertinib made by methods disclosed herein.

### RELATED APPLICATION DISCLOSURE

This application claims the benefit pursuant to 35 U.S.C. § 119 of European Patent Application No. 24181384.9, filed on 11 Jun. 2024, pending, which is hereby incorporated by reference herein in its entirety.

### FIELD OF THE INVENTION

The present invention relates to crystalline forms of zongertinib which are particularly suitable intermediates in large scale manufacturing.

### BACKGROUND OF THE INVENTION

Zongertinib is a selective inhibitor of wild type HER2 and mutant HER2 useful in the treatment and/or prevention of diseases and/or conditions wherein the inhibition of wild type and/or mutant HER2 is of therapeutic benefit, especially oncological and/or hyperproliferative diseases, such as cancer. It can be represented by the following chemical structure according to formula (1)

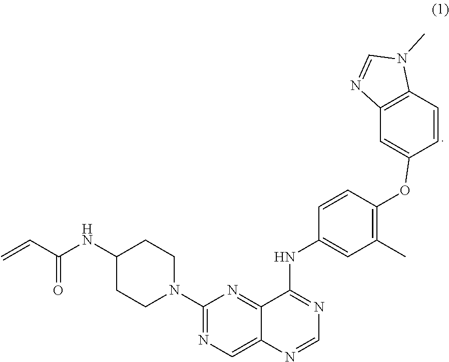 [SMILES: C=CC(=O)NC1CCN(c2ncc3ncnc(Nc4ccc(Oc5ccc6c(c5)ncn6C)c(C)c4)c3n2)CC1]

Zongertinib is disclosed in table 8 of WO 2021/213800 A1 as Compound I-01. The last step of its synthesis includes purification by preparative RP-HPLC-MS. The application does neither provide information about the physical properties of the obtained material nor mention a crystallization step.

Different solid-state forms of an active pharmaceutical ingredient often possess different properties. Differences in physicochemical properties, may translate into improved processing or handling during drug substance and drug product manufacturing e.g. pharmaceutical processes such as stirring, isolation, drying, mixing, milling, sieving etc. can benefit from altered physicochemical properties. Furthermore, chromatography can be avoided if a solid-state form with suitable properties is available.

There is thus a need for the provision of solid-state forms of zongertinib having advantageous physicochemical properties e.g. properties which allow for the reliable, fast and economic manufacturing of zongertinib and pharmaceutical compositions comprising zongertinib such as solid dispersions.

### SUMMARY OF THE INVENTION

According to a first aspect, the present invention provides crystalline forms of zongertinib, hereinafter also designated as form “XXIV”, form “XIX”, form “XVIII” and form “XX”.

It was surprisingly found, that forms XXIV, XIX and XX of the present invention are thermodynamically stable in various aqueous organic solvents. For example, form IV converted to form XXIV in aqueous isopropyl acetate and aqueous tetrahydrofuran, while form IV slurries in aqueous ethanol and aqueous acetonitrile lead to form XIX (see example 1), indicating the thermodynamic stability of forms XXIV and XIX in these solvent systems. Similarly, a mixture of forms III and IV fully transformed into form XX when stirred in pure water (see example 2.2) or in diluted aqueous isopropyl acetate (see example 2.1) again proofing the thermodynamic stability of form XX in these solvents. Organic solvents like acetonitrile, ethanol, isopropyl acetate and tetrahydrofuran and their respective mixtures with water are particularly suitable solvents employed in large-scale manufacturing, particularly in the last step(s) of a chemical synthesis. Hence, a crystalline form which is stable in these solvents allows for the reliable production of a defined crystalline form of zongertinib since phase changes can be excluded. Furthermore, the fact that form XX is a stable form in pure water opens the way for alternative formulations such as aqueous suspensions for oral or parenteral use.

In addition, crystalline forms XXIV and XIX of the present invention are characterized by lath-shaped and/or plate-shaped crystals (see example 7, FIG. 13 and FIG. 14). This favorable crystal habits translate into excellent isolation properties, in particular fast filtration times. While, for example, isolation of needle-shaped zongertinib crystals such as form XX (see example 7, FIG. 15) require extended filtration times, filtration times of forms XXIV and XIX are dramatically improved, particularly on industrial scale.

Moreover, isolation of form XXIV, form XIX or mixtures of both exhibits an improvement in chemical purity. Specifically, it provides an efficient purification of late-eluting impurities. Hence, forms XXIV and/or XIX are particularly suitable to deplete reaction impurities, particularly on industrial scale.

While reaction impurities are effectively depleted, forms XXIV, XIX contain significant amounts of organic solvents. However, upon drying organic solvents can be removed leading to another form designated form XVIII. Hence, having forms XIX and/or XXIV in hands, for the first time makes form XVIII accessible through solvent removal (see example 3). While the crystal shape is not affected by the drying process, Form XVIII is characterized by a significantly lower organic solvent content compared to forms XXIV and form XIX. Thus form XVIII possesses the same beneficial crystal habit but has the additional advantage of low organic solvent content compared to forms XXIV and XIX.

Hence, the crystalline forms of the present invention are particularly suitable forms for large scale manufacturing of zongertinib. They can for example be employed as intermediates to produce various solid-state forms of zongertinib, for the production of amorphous solid dispersions comprising zongertinib and a dispersion carrier, and/or for the manufacture of aqueous dosage forms.

As a further aspect, compositions comprising the crystalline forms XXIV, XIX, XVIII and XX of zongertinib are provided.

As yet another aspect, methods are provided for producing crystalline forms XXIV, XIX, XVIII and XX of zongertinib and compositions comprising the same. The crystalline forms of zongertinib obtained by or obtainable by such methods represent further aspects of the invention.

Still another aspect concerns the use of crystalline forms XXIV, XIX, XVIII and XX of zongertinib as intermediates to prepare various solid-state forms of zongertinib or compositions comprising solid-state forms of zongertinib.

Also provided herein is the use of crystalline forms XXIV, XIX, XVIII and XX of zongertinib to prepare a solid dispersion comprising zongertinib and a pharmaceutically acceptable dispersion carrier. A process of preparing a solid dispersion of zongertinib with the crystalline forms of the present invention is also provided.

### Abbreviation List

XRPD X-ray powder diffractogram
DSC differential scanning calorimetry
TGA thermogravimetric analysis
PLM polarized light microscopy
wt-% weight percent
vol volume
hrs hours
rpm rotations per minute
RH relative humidity
RT room temperature
ACN acetonitrile
IPAc isopropyl acetate
EtOH ethanol
THF tetrahydrofuran
DCM dichloromethane
BuOH 1-butanol
IPA isopropyl alcohol (2-propanol)
MeOH methanol

### Definitions

In the context of the present invention the following definitions have the indicated meaning, unless explicitly stated otherwise.

The term “zongertinib” as used herein, refers to a compound that is represented by the chemical structure according to formula (1)

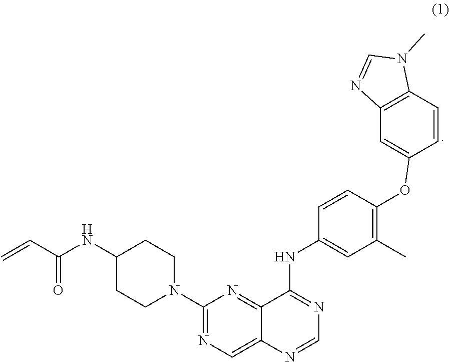 [SMILES: C=CC(=O)NC1CCN(c2ncc3ncnc(Nc4ccc(Oc5ccc6c(c5)ncn6C)c(C)c4)c3n2)CC1]

As used herein, the term “measured at a temperature in the range of from 20 to 30° C.” refers to a measurement under standard conditions. Typically, standard conditions mean a temperature in the range of from 20 to 30° C., i.e. at room temperature. Standard conditions can mean a temperature of about 22° C.

As used herein, the term “room temperature” refers to a temperature in the range of from 20 to 30° C.

The term “reflection” with regard to X-ray powder diffraction (XRPD) as used herein, means peaks in an X-ray diffractogram. These peaks are caused at certain diffraction angles (Bragg angles) by constructive interference from X-rays scattered by a specific set of parallel planes of atoms in solid material, which is distributed in an ordered and repetitive pattern in a long-range positional order. Such solid material is classified as crystalline material, whereas amorphous material is defined as solid material, which lacks long-range order and only displays short-range order, thus resulting in broad scattering. According to literature, long range order e.g. extends over approximately 100 to 1000 atoms, whereas short-range order is over a few atoms only (see “Fundamentals of Powder Diffraction and Structural Characterization of Materials” by Vitalij K. Pecharsky and Peter Y. Zavalij, Kluwer Academic Publishers, 2003, page 3).

The term “solid-state form” as used herein refers to any crystalline and/or amorphous phase of a compound.

The term “form IV” as used herein, when talking about a solid-state form of zongertinib refers to the crystalline form of zongertinib which is disclosed in WO 2024/133302 A1 and therein referred to as form IV. Form IV can be characterized by having an XRPD comprising reflections at 2-Theta angles of (5.9±0.2)°, (11.7±0.2)°, (14.7±0.2)°, (16.7±0.2)°, (18.8±0.2)° and (19.2±0.2), when measured at a temperature in the range of from 20 to 30° C. with Cu—Kα radiation at a wavelength of 1.54056 Å (Cu—Kα₁) or 1.54184 Å (Cu—Kα_{1,2}).

The term “form III” as used herein, when talking about a solid-state form of zongertinib refers to a crystalline form of zongertinib which is disclosed in WO 2024/133302 A1 and therein referred to as form III. Form III can be characterized by having an XRPD comprising reflections at 2-Theta angles of (6.2±0.2)°, (9.5±0.2)°, (11.4±0.2)°, (12.4±0.2)° and (16.2±0.2)°, when measured at a temperature in the range of from 20 to 30° C. with Cu—Kα radiation at a wavelength of 1.54056 Å (Cu—Kα₁) or 1.54184 Å (Cu—Kα_{1,2}).

The term “form I” as used herein, when talking about a solid-state form of zongertinib refers to a crystalline form of zongertinib which is disclosed in WO 2024/133302 A1 and therein referred to as form I. Form I can be characterized by having an XRPD comprising reflections at 2-Theta angles of (6.1±0.2)°, (7.9±0.2)°, (11.1±0.2)°, (12.0±0.2)°, (17.2±0.2)° and (17.9±0.2)°, when measured at a temperature in the range of from 20 to 30° C. with Cu—Kα radiation at a wavelength of 1.54056 Å (Cu—Kα₁) or 1.54184 Å (Cu—Kα_{1,2}).

The term “solvate” as used herein, refers to a crystalline solid where either solvent is cooperated in or accommodated by the crystal structure e.g. is part of the crystal structure or entrapped into the crystal (solvent inclusions). Thereby, solvent can be present in a stoichiometric or non-stoichiometric amount. When solvent is present in stoichiometric amount, the solvate may be referred to by adding greek numeral prefixes. For example, a solvate may be referred to as a hemisolvate or as a monosolvate depending on the solvent/API stoichiometry.

The term “hydrate” as used herein refers to a crystalline solid where either water is cooperated in or accommodated by the crystal structure e.g. is part of the crystal structure or entrapped into the crystal (water inclusions). Thereby, water can be present in a stoichiometric or non-stoichiometric amount. When water is present in stoichiometric amount, the hydrate may be referred to by adding greek numeral prefixes. For example, a hydrate may be referred to as a hemihydrate or as a monohydrate depending on the water/API stoichiometry.

As used herein, the term “plate-like” when describing the shape of a crystal refers to flat, tabular crystals which have similar breadth and width.

The term “lath-shaped” as used herein when describing the shape of a crystal refers to elongated/tabular, thin and blade-like crystals.

As used herein, the term “needle-shaped” when describing the shape of a crystal refers to acicular, thin and highly elongated crystals having similar width and breadth.

As used herein, the term “mother liquor” refers to the solution remaining after crystallization of a solid.

As used herein, the term “solid dispersion” refers to a system in a solid state comprising at least two components, wherein one component, such as zongertinib or generally an active pharmaceutical ingredient (API), preferably in amorphous state, is dispersed throughout another component such as a pharmaceutically acceptable solid dispersion carrier, particularly a dispersion polymer.

The phrase “pharmaceutically acceptable” is employed herein to refer to those compounds, materials, compositions, and/or dosage forms which are, within the scope of sound medical judgement, suitable for use in contact with the tissues of human beings without excessive toxicity, irritation, allergic response, or other problem or complication, and commensurate with a reasonable benefit/risk ratio.

As used herein, the term “dispersion carrier” refers to a carrier component that allows for an API such as zongertinib to be dispersed throughout such that a solid dispersion may form. In embodiments, zongertinib is dispersed at the molecular level in the dispersion carrier.

The term “spray drying” as used herein is used conventionally and broadly and generally refers to processes that involve the atomization of a solution, slurry, or emulsion containing one or more components of the desired product into droplets by spraying followed by the rapid evaporation of the sprayed droplets into solid powder by hot air at a certain temperature and pressure. Spray drying is a process known to a person skilled in the art.

### BRIEF DESCRIPTION OF THE FIGURES

FIG. 1: illustrates a representative XRPD of zongertinib form XXIV according to the present invention. The x-axis shows the scattering angle in °2-Theta, the y-axis shows the intensity of the scattered x-ray beam in counts of detected photons.

FIG. 2: illustrates a representative XRPD of zongertinib form XIX according to the present invention. The x-axis shows the scattering angle in °2-Theta, the y-axis shows the intensity of the scattered x-ray beam in counts of detected photons.

FIG. 3: illustrates a representative XRPD of zongertinib form XVIII according to the present invention. The x-axis shows the scattering angle in °2-Theta, the y-axis shows the intensity of the scattered x-ray beam in counts of detected photons.

FIG. 4: illustrates a representative XRPD of zongertinib form XX according to the present invention. The x-axis shows the scattering angle in °2-Theta, the y-axis shows the intensity of the scattered x-ray beam in counts of detected photons.

FIG. 5: illustrates a representative DSC curve of zongertinib form XXIV according to the present invention. The x-axis shows the temperature in degree Celsius (° C.), the y-axis shows the heat flow in Watt per gram (W/g) with exothermic peaks going up.

FIG. 6: illustrates a representative DSC curve of zongertinib form XIX according to the present invention. The x-axis shows the temperature in degree Celsius (° C.), the y-axis shows the heat flow in Watt per gram (W/g) with exothermic peaks going up.

FIG. 7: illustrates a representative DSC curve of zongertinib form XVIII according to the present invention. The x-axis shows the temperature in degree Celsius (° C.), the y-axis shows the heat flow in Watt per gram (W/g) with exothermic peaks going up.

FIG. 8: illustrates a representative DSC curve of zongertinib form XX according to the present invention. The x-axis shows the temperature in degree Celsius (° C.), the y-axis shows the heat flow in Watt per gram (W/g) with exothermic peaks going up.

FIG. 9: illustrates a representative TGA curve of zongertinib form XXIV according to the invention. The x-axis shows the temperature in degree Celsius (C), the y-axis the weight in percent (wt-%).

FIG. 10: illustrates a representative TGA curve of zongertinib form XIX according to the invention. The x-axis shows the temperature in degree Celsius (° C.), the y-axis the weight in percent (wt-%).

FIG. 11: illustrates a representative TGA curve of zongertinib form XVIII according to the invention. The x-axis shows the temperature in degree Celsius (° C.), the y-axis the weight in percent (wt-%).

FIG. 12: illustrates a representative TGA curve of zongertinib form XX according to the invention. The x-axis shows the temperature in degree Celsius (C), the y-axis the weight in percent (wt-%).

FIG. 13: illustrates a PLM image (magnification 20×) of zongertinib form XXIV crystals.

FIG. 14: illustrates a PLM image (magnification 20×) of zongertinib form XIX crystals.

FIG. 15: illustrates a PLM image (magnification 40×) of zongertinib form XX crystals.

### DETAILED DESCRIPTION OF THE INVENTION

The present invention provides crystalline forms of zongertinib, herein also designated as form “XXIV”, form “XIX”, form “XVIII” and form “XX”.

The crystalline forms of zongertinib of the present invention may be characterized by analytical methods well known in the field of the pharmaceutical industry for characterizing solids. Such methods comprise but are not limited to XRPD, DCS, TGA and PLM. The crystalline forms of the present invention may be characterized by one of the aforementioned methods or by combining two or more of them. In particular, the crystalline forms of zongertinib of the present invention may be characterized by any one of the following aspects and corresponding embodiments or by combining two or more of the corresponding embodiments.

### Form XXIV

In one aspect, the invention relates to a crystalline form of zongertinib (form XXIV) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.2)° and (19.3±0.2)°.

In one embodiment, the invention relates to a crystalline form of zongertinib (form XXIV) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):

$$( 5.8 \pm 0 \text{.2} ) ° , ( 7. \pm 0 \text{.2} ) ° , and ( 19.3 \pm 0 \text{.2} ) ° ; or$$

$$( 5.8 \pm 0 \text{.2} ) ° , ( 7. \pm 0 \text{.2} ) ° , ( 14. 1 \pm 0 \text{.2} ) ° , and ( 19.3 \pm 0 \text{.2} ) ° ; or$$

$$( 5.8 \pm 0 \text{.2} ) ° , ( 7. \pm 0 \text{.2} ) ° , ( 12. 2 \pm 0 \text{.2} ) ° , ( 14.1 \pm 0 \text{.2} ) ° , and ( 19.3 \pm 0 \text{.2} ) ° ; or$$

$$( 5.8 \pm 0 \text{.2} ) ° , ( 7. \pm 0 \text{.2} ) ° , ( 12.2 \pm 0 \text{.2} ) ° , ( 14.1 \pm 0 \text{.2} ) ° , ( 19. 3 \pm 0 \text{.2} ) ° , and ( 20.9 \pm 0 \text{.2} ) ° ; or$$

$$( 5.8 \pm 0 \text{.2} ) ° , ( 7. \pm 0 \text{.2} ) ° , ( 12.2 \pm 0 \text{.2} ) ° , ( 14.1 \pm 0 \text{.2} ) ° , ( 19.3 \pm 0 \text{.2} ) ° , ( 20. 0 \pm 0 \text{.2} ) ° , and ( 20.9 \pm 0.2 ) ° ; or$$

$$( 5.8 \pm 0 \text{.2} ) ° , ( 7. \pm 0 \text{.2} ) ° , ( 12.2 \pm 0 \text{.2} ) ° , ( 14.1 \pm 0 \text{.2} ) ° , ( 16. 3 \pm 0 \text{.2} ) ° , ( 19.3 \pm 0 \text{.2} ) ° , ( 20. \pm 0 \text{.2} ) ° , and ( 20.9 \pm 0 \text{.2} ) ° .$$

In another embodiment, the invention relates to a crystalline form of zongertinib (form XXIV) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (5.8±0.2)°, (7.0±0.2)°, (12.2±0.2)°, (14.1±0.2)°, (15.1±0.2)°, (16.3±0.2)°, (20.0±0.2)°, (20.9±0.2)°, (25.5±0.2)°, and (26.2±0.2)°.

In still another embodiment, the invention relates to a crystalline form of zongertinib (form XXIV) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.1)° and (19.3±0.1)°.

In a further embodiment, the invention relates to a crystalline form of zongertinib (form XXIV) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):

$$( 5.8 \pm 0.1 ) ° , ( 7. \pm 0.1 ) ° , and ( 19.3 \pm 0.1 ) ° ; or$$

$$( 5.8 \pm 0.1 ) ° , ( 7. \pm 0.1 ) ° , ( 14.1 \pm 0.1 ) ° , and ( 19.3 \pm 0.1 ) ° ; or$$

$$( 5.8 \pm 0.1 ) ° , ( 7. \pm 0.1 ) ° , ( 12.2 \pm 0.1 ) ° , ( 14.1 \pm 0.1 ) ° , and ( 19.3 \pm 0.1 ) ° ; or$$

$$( 5.8 \pm 0.1 ) ° , ( 7. \pm 0.1 ) ° , ( 12.2 \pm 0.1 ) ° , ( 14.1 \pm 0.1 ) ° , ( 19.3 \pm 0.1 ) ° , and ( 20.9 \pm 0.1 ) ° ; or$$

$$( 5.8 \pm 0.1 ) ° , ( 7. \pm 0.1 ) ° , ( 12.2 \pm 0.1 ) ° , ( 14.1 \pm 0.1 ) ° , ( 19.3 \pm 0.1 ) ° , ( 20. \pm 0.1 ) ° , and ( 20.9 \pm 0.1 ) ° ; or$$

$$( 5.8 \pm 0.1 ) ° , ( 7. \pm 0.1 ) ° , ( 12.2 \pm 0.1 ) ° , ( 14.1 \pm 0.1 ) ° , ( 16.3 \pm 0.1 ) ° , ( 19.3 \pm 0.1 ) ° , ( 20. \pm 0.1 ) ° , and ( 20.9 \pm 0.1 ) ° .$$

In another embodiment, the invention relates to a crystalline form of zongertinib (form XXIV) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (5.8±0.1)°, (7.0±0.1)°, (12.2±0.1)°, (14.1±0.1)°, (15.1±0.1)°, (16.3±0.1)°, (20.0±0.1)°, (20.9±0.1)°, (25.5±0.1)°, and (26.2±0.1)°.

In a particular embodiment, form XXIV is a solvate, a hydrate or any mixture thereof.

In still another embodiment, the invention relates to a crystalline form of zongertinib (form XXIV) characterized by having a DSC curve comprising an endotherm, preferably a first endotherm, having an onset at a temperature of (60±5)° C., preferably of (60±2)° C., such as 60° C., when measured at a temperature in the range of from 25 to 400° C. and a heating rate of 10° C./min. In another embodiment, the invention relates to a crystalline form of zongertinib (form XXIV) characterized by having a DSC curve comprising an endotherm, preferably a first endotherm, having a peak at a temperature of (80±5)° C., preferably of (80±2)° C., such as 80° C., when measured at a temperature in the range of from 25 to 400° C. and a heating rate of 10° C./min. In a particular embodiment, the endotherm is due to loss of solvent such as loss of organic solvent and/or water. In one embodiment, the organic solvent is selected from isopropyl acetate or tetrahydrofuran.

In yet another embodiment, the invention relates to a crystalline form of zongertinib (form XXIV) characterized by having a crystal habit comprising lath-shaped and/or plate-shaped crystals.

In another aspect, the invention relates to a composition comprising form XXIV of zongertinib as defined herein, wherein said composition is essentially free of a solid-state form of zongertinib other than form XXIV. For example, the invention relates to a composition comprising form XXIV of zongertinib as defined herein, wherein said composition comprises at most 20 wt-%, preferably at most 15 wt-%, more preferably at most 10 wt-%, most preferably at most 5, 4, 3, 2 or 1 wt-% of a solid-state form of zongertinib other than form XXIV, based on the weight of the composition. In one embodiment, the other solid-state form is form XIX as defined herein, amorphous zongertinib or any mixtures thereof.

In one embodiment, the invention relates to a composition comprising form XXIV of zongertinib as defined herein, wherein form XXIV of zongertinib is present in an amount of at least 80 wt-%, preferably of at least 85 wt-%, more preferably of at least 90 wt-%, including at least 90, 91, 92, 93, 94, 95, 96, 97, 98 or 99 wt-%, and also including equal to 100 wt-%, based on the weight of the composition. The remaining material may comprise solid-state form(s) of zongertinib other than form XXIV. Preferably, the remaining material is form XIX as defined herein or amorphous zongertinib.

In another aspect, the invention relates to a process for the preparation of form XXIV or the composition comprising form XXIV of zongertinib as defined herein, said process comprising:
(i) providing a crystalline form of zongertinib (form IV) characterized by having an XRPD comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at most 12 volumes of a solvent mixture comprising water and an organic solvent selected from isopropyl acetate or tetrahydrofuran at a temperature in the range of from 20 to 50° C.; and
(iii) isolating at least a part of the crystalline form of zongertinib or the composition comprising the crystalline form of zongertinib obtained in (ii).

The form IV starting material in (i) can be prepared according to any one of the procedures provided in reference example 1.3.

In one embodiment, the organic solvent/water ratio in (ii) is in the range of from 2:1 to 1:1 (vol:vol). For example, the isopropyl acetate/water ratio is 1:1 (vol:vol). In another embodiment, the tetrahydrofuran/water ratio is 2:1 (vol:vol). In a further embodiment, the amount of solvent applied in (ii) is 12 volumes. The temperature during slurrying in (ii) may be varied in the range of from 20 to 50° C. The variation may comprise heating cycles from 20 to 50° C. and cooling cycles from 50 to 20° C. The heating rate may be in the range of from 0.1° to 10° C./min, preferably of from 1 to 5° C./min. The cooling rate may be in the range of from −0.1° to −10° C./min, preferably of from −1 to −5° C./min. In one embodiment, the slurrying in (ii) comprises at least one heating cycle and at least one cooling cycle.

Once, the crystalline form XXIV of zongertinib of the present invention or the composition comprising the crystalline form XXIV of zongertinib of the present invention is obtained in sufficient amount, at least a part of the solid, preferably most of the solid, most preferably substantially all obtained solid is separated from the mother liquor in step (iii) of the above-described process. Thereby, the solid may be separated from the mother liquor by any conventional method known to the skilled person. In one embodiment, the solid is separated from the mother liquor by filtration, centrifugation, solvent evaporation and/or decantation. In a preferred embodiment, the solid is separated from the mother liquor by filtration and/or centrifugation. In a most preferred embodiment, the solid is separated from the mother liquor by filtration.

According to a further aspect, provided is a crystalline form or a composition comprising the crystalline form obtained or obtainable by:
(i) providing a crystalline form of zongertinib (form IV) characterized by having an XRPD comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at most 12 volumes of a solvent mixture comprising water and an organic solvent selected from isopropyl acetate or tetrahydrofuran at a temperature in the range of from 20 to 50° C.; and
(iii) isolating at least a part of the crystalline form of zongertinib or the composition comprising the crystalline form of zongertinib obtained in (ii).

In embodiments of this obtained or obtainable crystalline form or composition comprising the crystalline form, steps (ii) and (iii) can be performed as detailed above.

The crystalline form or the composition comprising the crystalline form obtainable or obtained by the process according to this aspect and its preferred embodiments is a further object of the invention. Preferably, this crystalline form is form XXIV as defined herein and may be characterized by having an XRPD comprising the reflections as defined above for form XXIV. Preferably, this crystalline form corresponds to form XXIV as defined herein in its broadest form or in any embodiment.

In still another aspect, the invention relates to the use of form XXIV of zongertinib as defined herein for the preparation of a solid-state form of zongertinib or a composition comprising a solid-state form of zongertinib. In one embodiment, the prepared solid-state form of zongertinib is selected from the group consisting of form III, form IV and form XVIII as defined herein. In yet another embodiment, the prepared solid-state form of zongertinib is amorphous. In still another embodiment, the prepared solid-state form of zongertinib is not form XXIV.

In one embodiment, the invention relates to the use of form XXIV of zongertinib as defined herein as an intermediate for the preparation of a solid-state form of zongertinib or a composition comprising a solid-state form of zongertinib. In one embodiment, the prepared solid-state form of zongertinib is selected from the group consisting of form III, form IV and form XVIII as defined herein. In yet another embodiment, the prepared solid-state form of zongertinib is amorphous. In still another embodiment, the prepared solid-state form of zongertinib is not form XXIV.

In still another embodiment, the invention relates to the use of form XXIV of zongertinib as defined herein for the preparation of a solid-state form of zongertinib other than form XXIV or a composition comprising a solid-state form of zongertinib other than form XXIV. In one embodiment, the other solid-state form of zongertinib is selected from the group consisting of form III, form IV and form XVIII as defined herein. In yet another embodiment, the other solid-state form of zongertinib is amorphous.

In yet another aspect, the invention relates to a process for the preparation of a solid-state form of zongertinib or a composition comprising a solid-state form of zongertinib comprising:
(i) dissolving or suspending form XXIV as defined herein in a suitable solvent or in a mixture of suitable solvents; and
(ii) crystallizing or precipitating a solid-state form of zongertinib or a composition comprising a solid-state form of zongertinib from the mixture provided in (i); and
(iii) isolating the solid obtained in (ii).

In one embodiment of the above defined process, the prepared solid-state form of zongertinib is selected from the group consisting of form III, form IV and form XVIII as defined herein. In yet another embodiment, the prepared solid-state form of zongertinib is amorphous. In still another embodiment, the prepared solid-state form of zongertinib is not form XXIV.

### Form XIX

In one aspect, the invention relates to a crystalline form of zongertinib (form XIX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (8.3±0.2)° and (14.6±0.2)°.

In one embodiment, the invention relates to a crystalline form of zongertinib (form XIX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):

$$( 7. \pm 0.2 ) ° , ( 8.3 \pm 0 \text{.2} ) ° , and ( 14.6 \pm 0 \text{.2} ) ° ; or$$

$$( 5.7 \pm 0 \text{.2} ) ° , ( 7. \pm 0.2 ) ° , ( 8.3 \pm 0 \text{.2} ) ° , and ( 14.6 \pm 0 \text{.2} ) ° ; or$$

$$( 5.7 \pm 0 \text{.2} ) ° , ( 7. \pm 0.2 ) ° , ( 8.3 \pm 0 \text{.2} ) ° , ( 14. 6 \pm 0 \text{.2} ) ° , and ( 16.6 \pm 0 \text{.2} ) ° ; or$$

$$( 5.7 \pm 0 \text{.2} ) ° , ( 7. \pm 0.2 ) ° , ( 8.3 \pm 0 \text{.2} ) ° , ( 13. 5 \pm 0 \text{.2} ) ° , ( 14. 6 \pm 0 \text{.2} ) ° , and ( 16.6 \pm 0 \text{.2} ) ° ; or$$

$$( 5.7 \pm 0 \text{.2} ) ° , ( 7. \pm 0.2 ) ° , ( 8.3 \pm 0 \text{.2} ) ° , ( 12. 0 \pm 0 \text{.2} ) ° , ( 13. 5 \pm 0 \text{.2} ) ° , ( 14. 6 \pm 0 \text{.2} ) ° , and ( 16.6 \pm 0.2 ) ° ; or$$

$$( 5.7 \pm 0 \text{.2} ) ° , ( 7. \pm 0.2 ) ° , ( 7.9 \pm 0 \text{.2} ) ° , ( 8.3 \pm 0 \text{.2} ) ° , ( 12. 0 \pm 0 \text{.2} ) ° , ( 13. 5 \pm 0 \text{.2} ) ° , ( 14. 6 \pm 0 \text{.2} ) ° , and ( 16.6 \pm 0 \text{.2} ) ° ; or$$

$$( 5.7 \pm 0 \text{.2} ) ° , ( 7. \pm 0.2 ) ° , ( 7.9 \pm 0 \text{.2} ) ° , ( 8.3 \pm 0 \text{.2} ) ° , ( 12. 0 \pm 0 \text{.2} ) ° , ( 12. 9 \pm 0 \text{.2} ) ° , ( 13. 5 \pm 0 \text{.2} ) ° , ( 14.6 \pm 0 \text{.2} ) ° , and ( 16.6 \pm 0 \text{.2} ) ° ; or$$

$$( 5.7 \pm 0 \text{.2} ) ° , ( 7. \pm 0.1 ) ° , ( 7.9 \pm 0 \text{.2} ) ° , ( 8.3 \pm 0 \text{.2} ) ° , ( 10. 3 \pm 0 \text{.2} ) ° , ( 12. 0 \pm 0 \text{.2} ) ° , ( 12. 9 \pm 0 \text{.2} ) ° , ( 13.5 \pm 0.2 ) ° , ( 14. 6 \pm 0 \text{.2} ) ° , and ( 16.6 \pm 0 \text{.2} ) ° .$$

In another embodiment, the invention relates to a crystalline form of zongertinib (form XIX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.2)°, (8.3±0.2)°, (13.5±0.2)°, (14.6±0.2)°, (16.6±0.2)°, (17.6±0.2)°, (22.8±0.2)°, (24.1±0.2)°, (24.4±0.2)°, and (27.3±0.2)°.

In still another embodiment, the invention relates to a crystalline form of zongertinib (form XIX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (8.3±0.1)° and (14.6±0.1)°.

In a further embodiment, the invention relates to a crystalline form of zongertinib (form XIX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):

$$( 7. \pm 0 \text{.1} ) ° , ( 8.3 \pm 0 \text{.1} ) ° , and ( 14.6 \pm 0 \text{.1} ) ° ; or$$

$$( 5.7 \pm 0 \text{.1} ) ° , ( 7. \pm 0 \text{.1} ) ° , ( 8.3 \pm 0 \text{.1} ) ° , and ( 14.6 \pm 0 \text{.1} ) ° ; or$$

$$( 5.7 \pm 0 \text{.1} ) ° , ( 7. \pm 0 \text{.1} ) ° , ( 8.3 \pm 0 \text{.1} ) ° , ( 14.6 \pm 0 \text{.1} ) ° , and ( 16.6 \pm 0 \text{.1} ) ° ; or$$

$$( 5.7 \pm 0 \text{.1} ) ° , ( 7. \pm 0 \text{.1} ) ° , ( 8.3 \pm 0 \text{.1} ) ° , ( 13. 5 \pm 0 \text{.1} ) ° , ( 14.6 \pm 0 \text{.1} ) ° , and ( 16.6 \pm 0 \text{.1} ) ° ; or$$

$$( 5.7 \pm 0 \text{.1} ) ° , ( 7. \pm 0 \text{.1} ) ° , ( 8.3 \pm 0 \text{.1} ) ° , ( 12. 0 \pm 0 \text{.1} ) ° , ( 13. 5 \pm 0 \text{.1} ) ° , ( 14.6 \pm 0 \text{.1} ) ° , and ( 16.6 \pm 0 \text{.1} ) ° ; or$$

$$( 5.7 \pm 0 \text{.1} ) ° , ( 7. \pm 0 \text{.1} ) ° , ( 7.9 \pm 0 \text{.1} ) ° , ( 8.3 \pm 0 \text{.1} ) ° , ( 12. 0 \pm 0 \text{.1} ) ° , ( 13. 5 \pm 0 \text{.1} ) ° , ( 14.6 \pm 0 \text{.1} ) ° , and ( 16.6 \pm 0 \text{.1} ) ° ; or$$

$$( 5.7 \pm 0 \text{.1} ) ° , ( 7. \pm 0 \text{.1} ) ° , ( 7.9 \pm 0 \text{.1} ) ° , ( 8.3 \pm 0 \text{.1} ) ° , ( 12. 0 \pm 0 \text{.1} ) ° , ( 12. 9 \pm 0 \text{.1} ) ° , ( 13. 5 \pm 0 \text{.1} ) ° , ( 14.6 \pm 0.1 ) ° , and ( 16.6 \pm 0 \text{.1} ) ° ; or$$

$$( 5.7 \pm 0 \text{.1} ) ° , ( 7. \pm 0 \text{.1} ) ° , ( 7.9 \pm 0 \text{.1} ) ° , ( 8.3 \pm 0 \text{.1} ) ° , ( 10.3 \pm 0 \text{.1} ) ° , ( 12. 0 \pm 0 \text{.1} ) ° , ( 12. 9 \pm 0.1 ) ° , ( 13.5 \pm 0 \text{.1} ) ° , ( 14.6 \pm 0 \text{.1} ) ° , and ( 16.6 \pm 0 \text{.1} ) ° .$$

In another embodiment, the invention relates to a crystalline form of zongertinib (form XIX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.1)°, (8.3±0.1)°, (13.5±0.1)°, (14.6±0.1)°, (16.6±0.1)°, (17.6±0.1)°, (22.8±0.1)°, (24.1±0.1)°, (24.4±0.1)°, and (27.3±0.1)°.

In a particular embodiment, form XIX is a solvate, a hydrate or any mixture thereof.

In still another embodiment, the invention relates to a crystalline form of zongertinib (form XIX) characterized by having a DSC curve comprising an endotherm, preferably a first endotherm, having an onset at a temperature of (69±5)° C., when measured at a temperature in the range of from 25 to 400° C. and a heating rate of 10° C./min. In another embodiment, the invention relates to a crystalline form of zongertinib (form XIX) characterized by having a DSC curve comprising an endotherm, preferably a first endotherm, having a peak at a temperature of (92±5)° C., when measured at a temperature in the range of from 25 to 400° C. and a heating rate of 10° C./min. In a particular embodiment, the endothermic peak is due to loss of solvent and/or water. In one embodiment, the organic solvent is selected from ethanol or acetonitrile.

In yet another embodiment, the invention relates to a crystalline form of zongertinib (form XIX) characterized by having a crystal habit comprising lath-shaped crystals.

In another aspect, the invention relates to a composition comprising form XIX of zongertinib as defined herein, wherein said composition is essentially free of a solid-state form of zongertinib other than form XIX. For example, the invention relates to a composition comprising form XIX of zongertinib as defined herein, wherein said composition comprises at most 20 wt-%, preferably at most 15 wt-%, more preferably at most 10 wt-%, most preferably at most 5, 4, 3, 2 or 1 wt-% of a solid-state form of zongertinib other than form XIX, based on the weight of the composition. In one embodiment, the other solid-state form is form XXIV as defined herein, amorphous zongertinib or any mixtures thereof.

In one embodiment, the invention relates to a composition comprising form XIX of zongertinib as defined herein, wherein form XIX of zongertinib is present in an amount of at least 80 wt-%, preferably of at least 85 wt-%, more preferably of at least 90 wt-%, including at least 90, 91, 92, 93, 94, 95, 96, 97, 98 or 99 wt-%, and also including equal to 100 wt-%, based on the weight of the composition. The remaining material may comprise other solid-state form(s) of zongertinib. In one embodiment, the remaining material is form XXIV as defined herein or amorphous zongertinib.

In another aspect, the invention relates to a process for the preparation of form XIX or the composition comprising form XIX of zongertinib as defined herein, said process comprising:
(i) providing a crystalline form of zongertinib (form IV) characterized by having an XRPD comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at most 12 volumes of a solvent mixture comprising water and an organic solvent selected from ethanol or acetonitrile at a temperature in the range of from 20 to 50° C.; and
(iii) isolating at least a part of the crystalline form of zongertinib or the composition comprising the crystalline form of zongertinib obtained in (ii).

The form IV starting material in (i) can be prepared according to any one of the procedures provided in reference example 1.3.

In one embodiment, the organic solvent/water ratio in (ii) is 1:1 (vol:vol). For example, the ethanol/water ratio is 1:1 (vol:vol). In another example, the acetonitrile/water ratio is 1:1 (vol:vol). In a further embodiment, the amount of solvent applied in (ii) is 12 volumes. The temperature during slurrying in (ii) may be varied in the range of from 20 to 50° C. The variation may comprise heating cycles from 20 to 50° C. and cooling cycles from 50 to 20° C. The heating rate may be in the range of from 0.1° to 10° C./min, preferably of from 1 to 5° C./min. The cooling rate may be in the range of from −0.1° to −10° C./min, preferably of from −1 to −5° C./min. In one embodiment, the slurrying in (ii) comprises at least one heating cycle and at least one cooling cycle.

Once, the crystalline form XIX of zongertinib of the present invention or the composition comprising the crystalline form XIX of zongertinib of the present invention is obtained in sufficient amount, at least a part of the solid, preferably most of the solid, most preferably substantially all the solid is separated from the mother liquor in step (iii) of the above-described process. Thereby, the solid may be separated from the mother liquor by any conventional method known to the skilled person. In one embodiment, the solid is separated from the mother liquor by filtration, centrifugation, solvent evaporation and/or decantation. In a preferred embodiment, the solid is separated from the mother liquor by filtration and/or centrifugation. In a most preferred embodiment, the solid is separated from the mother liquor by filtration.

According to a further aspect, provided is a crystalline form or a composition comprising the crystalline form obtained or obtainable by:
(i) providing a crystalline form of zongertinib (form IV) characterized by having an XRPD comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at most 12 volumes of a solvent mixture comprising water and an organic solvent selected from ethanol or acetonitrile at a temperature in the range of from 20 to 50° C.; and
(iii) isolating at least a part of the crystalline form of zongertinib or the composition comprising the crystalline form of zongertinib obtained in (ii).

In embodiments of this obtained or obtainable crystalline form or composition comprising the crystalline form, steps (ii) and (iii) can be performed as detailed above.

The crystalline form or the composition comprising the crystalline form obtainable or obtained by the process according to this aspect and its preferred embodiments is a further object of the invention. Preferably, this crystalline form is form XIX as defined herein and may be characterized by having an XRPD comprising the reflections as defined above for form XIX. Preferably, this crystalline form corresponds to form XIX as defined herein in its broadest form or in any embodiment.

In still another aspect, the invention relates to the use of form XIX of zongertinib as defined herein for the preparation of a solid-state form of zongertinib or a composition comprising a solid-state form of zongertinib. In one embodiment, the prepared solid-state form of zongertinib is selected from the group consisting of form III, form IV and form XVIII as defined herein. In yet another embodiment, the prepared solid-state form of zongertinib is amorphous. In still another embodiment, the prepared solid-state form of zongertinib is not form XIX.

In one embodiment, the invention relates to the use of form XIX of zongertinib as defined herein as an intermediate for the preparation of a solid-state form of zongertinib or a composition comprising a solid-state form of zongertinib. In one embodiment, the prepared solid-state form of zongertinib is selected from the group consisting of form III, form IV and form XVIII as defined herein. In yet another embodiment, the prepared solid-state form of zongertinib is amorphous. In still another embodiment, the prepared solid-state form of zongertinib is not form XIX.

In still another embodiment, the invention relates to the use of form XIX of zongertinib as defined herein for the preparation of a solid-state form of zongertinib other than form XIX or a composition comprising a solid-state form of zongertinib other than form XIX. In one embodiment, the other solid-state form of zongertinib is selected from the group consisting of form III, form IV and form XVIII as defined herein. In yet another embodiment, the other solid-state form of zongertinib is amorphous.

In yet another aspect, the invention relates to a process for the preparation of a solid-state form of zongertinib or a composition comprising a solid-state form of zongertinib comprising:
(i) dissolving or suspending form XIX as defined herein in a suitable solvent or in a mixture of suitable solvents; and
(ii) crystallizing or precipitating a solid-state form of zongertinib or a composition comprising a solid-state form of zongertinib from the mixture provided in (i); and
(iii) isolating the solid obtained in (ii).

In one embodiment of the above defined process, the prepared solid-state form of zongertinib is selected from the group consisting of form III, form IV and form XVIII as defined herein. In yet another embodiment, the prepared solid-state form of zongertinib is amorphous. In still another embodiment, the prepared solid-state form of zongertinib is not form XIX.

### Form XVIII

In one aspect, the invention relates to a crystalline form of zongertinib (form XVIII) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.2±0.2)°, (6.8±0.2)° and (8.1±0.2)°.

In one embodiment, the invention relates to a crystalline form of zongertinib (form XVIII) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):

$$( 5.8 \pm 0.2 ) ° , ( 6.2 \pm 0 \text{.2} ) ° , ( 6.8 \pm 0 \text{.2} ) ° , and ( 8.1 \pm 0 \text{.2} ) ° ; or$$

$$( 5.8 \pm 0.2 ) ° , ( 6.2 \pm 0 \text{.2} ) ° , ( 6.8 \pm 0 \text{.2} ) ° , ( 8.1 \pm 0 \text{.2} ) ° , and ( 13.6 \pm 0 \text{.2} ) ° ; or$$

$$( 5.8 \pm 0.2 ) ° , ( 6.2 \pm 0 \text{.2} ) ° , ( 6.8 \pm 0 \text{.2} ) ° , ( 8.1 \pm 0 \text{.2} ) ° , ( 13.6 \pm 0 \text{.2} ) ° , and ( 16.7 \pm 0 \text{.2} ) ° ; or$$

$$( 5.8 \pm 0.2 ) ° , ( 6.2 \pm 0 \text{.2} ) ° , ( 6.8 \pm 0 \text{.2} ) ° , ( 8.1 \pm 0 \text{.2} ) ° , ( 12.7 \pm 0 \text{.2} ) ° , ( 13.6 \pm 0 \text{.2} ) ° , and ( 16.7 \pm 0 \text{.2} ) ° ; or$$

$$( 5.8 \pm 0.2 ) ° , ( 6.2 \pm 0 \text{.2} ) ° , ( 6.8 \pm 0 \text{.2} ) ° , ( 8.1 \pm 0 \text{.2} ) ° , ( 12.7 \pm 0 \text{.2} ) ° , ( 13.6 \pm 0 \text{.2} ) ° , ( 16.7 \pm 0 \text{.2} ) ° , and ( 20.1 \pm 0 \text{.2} ) ° ; or$$

$$( 5.8 \pm 0.2 ) ° , ( 6.2 \pm 0 \text{.2} ) ° , ( 6.8 \pm 0 \text{.2} ) ° , ( 8.1 \pm 0 \text{.2} ) ° , ( 12.7 \pm 0 \text{.2} ) ° , ( 13.6 \pm 0 \text{.2} ) ° , ( 16.7 \pm 0 \text{.2} ) ° , ( 20.1 \pm 0 \text{.2} ) ° , and ( 22. \pm 0 \text{.2} ) ° ; or$$

$$( 5.8 \pm 0.2 ) ° , ( 6.2 \pm 0 \text{.2} ) ° , ( 6.8 \pm 0 \text{.2} ) ° , ( 8.1 \pm 0 \text{.2} ) ° , ( 12.7 \pm 0 \text{.2} ) ° , ( 13.6 \pm 0 \text{.2} ) ° , ( 16.7 \pm 0 \text{.2} ) ° , ( 20.1 \pm 0 \text{.2} ) ° , ( 22. \pm 0 \text{.2} ) ° , and ( 24.2 \pm 0 \text{.2} ) ° .$$

In still another embodiment, the invention relates to a crystalline form of zongertinib (form XVIII) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.2±0.1)°, (6.8±0.1)° and (8.1±0.1)°.

In a further embodiment, the invention relates to a crystalline form of zongertinib (form XVIII) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):

$$( 5.8 \pm 0 \text{.1} ) ° , ( 6.2 \pm 0 \text{.1} ) ° , ( 6.8 \pm 0 \text{.1} ) ° , and ( 8.1 \pm 0 \text{.1} ) ° ; or$$

$$( 5.8 \pm 0 \text{.1} ) ° , ( 6.2 \pm 0 \text{.1} ) ° , ( 6.8 \pm 0 \text{.1} ) ° , ( 8.1 \pm 0 \text{.1} ) ° , and ( 13.6 \pm 0 \text{.1} ) ° ; or$$

$$( 5.8 \pm 0 \text{.1} ) ° , ( 6.2 \pm 0 \text{.1} ) ° , ( 6.8 \pm 0 \text{.1} ) ° , ( 8.1 \pm 0 \text{.1} ) ° , ( 13.6 \pm 0 \text{.1} ) ° , and ( 16.7 \pm 0.1 ) ° ; or$$

$$( 5.8 \pm 0 \text{.1} ) ° , ( 6.2 \pm 0 \text{.1} ) ° , ( 6.8 \pm 0 \text{.1} ) ° , ( 8.1 \pm 0 \text{.1} ) ° , ( 12.7 \pm 0 \text{.1} ) ° , ( 13.6 \pm 0.1 ) ° , and ( 16.7 \pm 0.1 ) ° ; or$$

$$( 5.8 \pm 0 \text{.1} ) ° , ( 6.2 \pm 0 \text{.1} ) ° , ( 6.8 \pm 0 \text{.1} ) ° , ( 8.1 \pm 0 \text{.1} ) ° , ( 12.7 \pm 0 \text{.1} ) ° , ( 13.6 \pm 0 \text{.1} ) ° , ( 16.7 \pm 0 \text{.1} ) ° , and ( 20.1 \pm 0 \text{.1} ) ° ; or$$

$$( 5.8 \pm 0 \text{.1} ) ° , ( 6.2 \pm 0 \text{.1} ) ° , ( 6.8 \pm 0 \text{.1} ) ° , ( 8.1 \pm 0 \text{.1} ) ° , ( 12.7 \pm 0 \text{.1} ) ° , ( 13. 6 \pm 0 \text{.1} ) ° , ( 16.7 \pm 0 \text{.1} ) ° , ( 20.1 \pm 0 \text{.1} ) ° , and ( 22. \pm 0 \text{.1} ) ° ; or$$

$$( 5.8 \pm 0 \text{.1} ) ° , ( 6.2 \pm 0 \text{.1} ) ° , ( 6.8 \pm 0 \text{.1} ) ° , ( 8.1 \pm 0 \text{.1} ) ° , ( 12. 7 \pm 0 \text{.1} ) ° , ( 13.6 \pm 0 \text{.1} ) ° , ( 16. 7 \pm 0 \text{.1} ) ° , ( 20.1 \pm 0 \text{.1} ) ° , ( 22. 0 \pm 0 \text{.1} ) ° , and ( 24.2 \pm 0 \text{.1} ) ° .$$

In still another embodiment, the invention relates to a crystalline form of zongertinib (form XVIII) characterized by having a DSC curve comprising an endotherm, preferably a first endotherm, having an onset at a temperature of (49±5)° C., preferably of (49±2)° C., such as 49° C., when measured at a temperature in the range of from 25 to 300° C. and a heating rate of 5° C./min. In another embodiment, the invention relates to a crystalline form of zongertinib (form XVIII) characterized by having a DSC curve comprising an endotherm, preferably a first endotherm, having a peak at a temperature of (80±5)° C., preferably of (80±2), such as 80° C., when measured at a temperature in the range of from 25 to 300° C. and a heating rate of 5° C./min. In a particular embodiment, the endothermic peak is due to loss of water.

In yet another embodiment, the invention relates to a crystalline form of zongertinib (form XVIII) characterized by having a crystal habit comprising lath-shaped and/or plate-shaped crystals.

In another aspect, the invention relates to a composition comprising form XVIII of zongertinib as defined herein, wherein said composition is essentially free of a solid-state form of zongertinib other than form XVIII. For example, the invention relates to a composition comprising form XVIII of zongertinib as defined herein, wherein said composition comprises at most 20 wt-%, preferably at most 15 wt-%, more preferably at most 10 wt-%, most preferably at most 5, 4, 3, 2 or 1 wt-% of a solid-state form of zongertinib other than form XVIII, based on the weight of the composition. In one embodiment, the other solid-state form is form XIX as defined herein, form XXIV as defined herein, amorphous zongertinib or any mixtures thereof.

In one embodiment, the invention relates to a composition comprising form XVIII of zongertinib as defined herein, wherein form XVIII is present in an amount of at least 80 wt-%, preferably of at least 85 wt-%, more preferably of at least 90 wt-%, including at least 90, 91, 92, 93, 94, 95, 96, 97, 98 or 99 wt-%, and also including equal to 100 wt-%, based on the weight of the composition. The remaining material may comprise other solid-state form(s) of zongertinib such as form XXIV, form XIX, amorphous or any mixtures thereof.

In another aspect, the invention relates to a process for the preparation of form XVIII of zongertinib or the composition comprising form XVIII as defined herein, said process comprising:
(i) providing zongertinib form XIX or XXIV, or a composition comprising form XIX or form XXIV as defined herein, or any mixture thereof as a solid; and
(ii) at least partially removing the solvent from the crystalline form, composition or mixture provided in (i).

The starting materials in step (i) of the above-described process can be prepared according to the teachings and examples of the present invention e.g. according to example 1 hereinafter.

The crystal structures of both, form XIX and form XXIV contain solvent such as organic solvent and water. Having forms XIX and/or XXIV in hands, for the first time makes form XVIII accessible through at least partial, preferably complete removal of organic solvent and/or water from forms XIX and/or XXIV. Solvent removal in step (ii) of the above-described process may be achieved by exposing forms XIX and/or XXIV to elevated temperature. In one embodiment, the elevated temperature is in the range of from 50 to 120° C., preferably of from 70 to 120° C., more preferably of from 80 to about 120° C. and most preferably of from 90 to 110° C. In another embodiment, forms XIX and/or XXIV are exposed to the elevated temperature for a period in the range of from 2 hours to 7 days, preferably of from 6 to 48 hours and most preferably of from 12 to 24 hours. In another embodiment, form XIX and/or form XXIV are additionally exposed to a vacuum for example at or below 200 mbar, 100 mbar, 50 mbar, or 20 mbar to remove the organic solvent and/or water.

According to a further aspect, provided is a crystalline form or a composition comprising the crystalline form obtained or obtainable by:
(i) providing zongertinib form XIX or form XXIV, or a composition comprising form XIX or form XXIV as defined herein, or any mixture thereof as a solid; and
(ii) at least partially removing the solvent from the crystalline form, composition or mixture provided in (i).

In embodiments of this obtained or obtainable crystalline form or the composition comprising the crystalline form, step (ii) can be performed as detailed above.

The crystalline form or the composition comprising the crystalline form obtainable or obtained by the process according to this aspect and its preferred embodiments is a further object of the invention. Preferably, this crystalline form is form XVIII as defined herein and may be characterized by having an XRPD comprising the reflections as defined above for form XVIII. Preferably, this crystalline form corresponds to form XVIII as defined herein in its broadest form or in any embodiment.

In still another aspect, the invention relates to the use of form XVIII of zongertinib as defined herein for the preparation of a solid-state form of zongertinib or a composition comprising a solid-state form of zongertinib. In one embodiment, the prepared solid-state form of zongertinib is selected from the group consisting of form III and form IV as defined herein. In yet another embodiment, the prepared solid-state form of zongertinib is amorphous.

In one embodiment, the invention relates to the use of form XVIII of zongertinib as defined herein as an intermediate for the preparation of a solid-state form of zongertinib or a composition comprising a solid-state form of zongertinib. In one embodiment, the prepared solid-state form of zongertinib is selected from the group consisting of form III and form IV as defined herein. In yet another embodiment, the prepared solid-state form of zongertinib is amorphous.

In still another embodiment, the invention relates to the use of form XVIII of zongertinib as defined herein for the preparation of a solid-state form of zongertinib other than form XVIII or a composition comprising a solid-state form of zongertinib other than form XVIII. In one embodiment, the other solid-state form of zongertinib is selected from the group consisting of form III and form IV as defined herein. In yet another embodiment, the other solid-state form of zongertinib is amorphous.

In yet another aspect, the invention relates to a process for the preparation of a solid-state form of zongertinib or a composition comprising a solid-state form of zongertinib comprising:
(i) dissolving or suspending form XVIII as defined herein in a suitable solvent or in a mixture of suitable solvents; and
(ii) crystallizing or precipitating a solid-state form of zongertinib or a composition comprising a solid-state form of zongertinib from the mixture provided in (i); and
(iii) isolating the solid obtained in (ii).

In one embodiment of the above defined process, the prepared solid-state form of zongertinib is selected from the group consisting of form III and form IV as defined herein. In yet another embodiment, the prepared solid-state form of zongertinib is amorphous. In still another embodiment, the prepared solid-state form of zongertinib is not form XVIII.

### Form XX

In one aspect, the invention relates to a crystalline form of zongertinib (form XX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.7±0.2)° and (8.8±0.2)°.

In one embodiment, the invention relates to a crystalline form of zongertinib (form XXIV) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):

$$( 6.7 \pm 0 \text{.2} ) ° , ( 8.8 \pm 0.2 ) ° , and ( 10.9 \pm 0 \text{.2} ) ° ; or$$

$$( 6.7 \pm 0 \text{.2} ) ° , ( 8.8 \pm 0 \text{.2} ) ° , ( 10.9 \pm 0 \text{.2} ) ° , and ( 14.1 \pm 0.2 ) ° ; or$$

$$( 6.7 \pm 0 \text{.2} ) ° , ( 8.8 \pm 0 \text{.2} ) ° , ( 10.9 \pm 0 \text{.2} ) ° , ( 13.3 \pm 0 \text{.2} ) ° , ( 14.1 \pm 0.2 ) ° ; or$$

$$( 6.7 \pm 0 \text{.2} ) ° , ( 8.8 \pm 0 \text{.2} ) ° , ( 10.9 \pm 0 \text{.2} ) ° , ( 13.3 \pm 0 \text{.2} ) ° , ( 14.1 \pm 0.2 ) ° , and ( 20.3 \pm 0 \text{.2} ) ° ; or$$

$$( 6.7 \pm 0 \text{.2} ) ° , ( 8.8 \pm 0 \text{.2} ) ° , ( 10.1 \pm 0 \text{.2} ) ° , ( 10.9 \pm 0 \text{.2} ) ° , ( 13.3 \pm 0 \text{.2} ) ° , ( 14.1 \pm 0.2 ) ° , and ( 20.3 \pm 0 \text{.2} ) ° ; or$$

$$( 6.7 \pm 0 \text{.2} ) ° , ( 8.8 \pm 0 \text{.2} ) ° , ( 10. 1 \pm 0 \text{.2} ) ° , ( 10. 9 \pm 0 \text{.2} ) ° , ( 12. \pm 0 \text{.2} ) ° , ( 13. 3 \pm 0 \text{.2} ) ° , ( 14.1 \pm 0.2 ) ° , and ( 20.3 \pm 0 \text{.2} ) ° .$$

In another embodiment, the invention relates to a crystalline form of zongertinib (form XX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.7±0.2)°, (8.8±0.2)°, (10.9±0.2)°, (13.3±0.2)°, (14.1±0.2)°, (16.5±0.2)°, (18.8±0.2)°, (20.3±0.2)°, (20.4±0.2)°, and (25.6±0.2).

In still another embodiment, the invention relates to a crystalline form of zongertinib (form XX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.7±0.1)° and (8.8±0.1)°.

In a further embodiment, the invention relates to a crystalline form of zongertinib (form XX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):

$$( 6.7 \pm 0 \text{.1} ) ° , ( 8.8 \pm 0 \text{.1} ) ° , and ( 10.9 \pm 0 \text{.1} ) ° ; or$$

$$( 6.7 \pm 0 \text{.1} ) ° , ( 8.8 \pm 0 \text{.1} ) ° , ( 10.9 \pm 0 \text{.1} ) ° , and ( 14.1 \pm 0 \text{.1} ) ° ; or$$

$$( 6.7 \pm 0 \text{.1} ) ° , ( 8.8 \pm 0 \text{.1} ) ° , ( 10.9 \pm 0 \text{.1} ) ° , ( 13.3 \pm 0 \text{.1} ) ° , and ( 14.1 \pm 0 \text{.1} ) ° ; or$$

$$( 6.7 \pm 0 \text{.1} ) ° , ( 8.8 \pm 0 \text{.1} ) ° , ( 10.9 \pm 0 \text{.1} ) ° , ( 13.3 \pm 0 \text{.1} ) ° , ( 14.1 \pm 0 \text{.1} ) ° , and ( 20.3 \pm 0.1 ) ° ; or$$

$$( 6.7 \pm 0 \text{.1} ) ° , ( 8.8 \pm 0 \text{.1} ) ° , ( 10.1 \pm 0 \text{.1} ) ° , ( 10.9 \pm 0 \text{.1} ) ° , ( 13.3 \pm 0 \text{.1} ) ° , ( 14.1 \pm 0 \text{.1} ) ° , and ( 20.3 \pm 0.1 ) ° ; or$$

$$( 6.7 \pm 0 \text{.1} ) ° , ( 8.8 \pm 0 \text{.1} ) ° , ( 10. 1 \pm 0 \text{.1} ) ° , ( 10. 9 \pm 0 \text{.1} ) ° , ( 12. 0 \pm 0 \text{.1} ) ° , ( 13. 3 \pm 0 \text{.1} ) ° , ( 14. 1 \pm 0 \text{.1} ) ° , and ( 20.3 \pm 0.1 ) ° .$$

In another embodiment, the invention relates to a crystalline form of zongertinib (form XX) characterized by having an XRPD comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.7±0.1)°, (8.8±0.1)°, (10.9±0.1)°, (13.3±0.1)°, (14.1±0.1)°, (16.5±0.1)°, (18.8±0.1)°, (20.3±0.1)°, (20.4±0.1)°.

In a particular embodiment, form XX is a solvate, a hydrate or any mixture thereof.

In still another embodiment, the invention relates to a crystalline form of zongertinib (form XX) characterized by having a DSC curve comprising an endotherm, preferably a first endotherm, having an onset at a temperature of (48±5)° C., preferably of (48±2)° C., such as 48° C., when measured at a temperature in the range of from 25 to 300° C. and a heating rate of 2° C./min. In another embodiment, the invention relates to a crystalline form of zongertinib (form XX) characterized by having a DSC curve comprising an endotherm, preferably a first endotherm, having a peak at a temperature of (65±5)° C., preferably of (65±2)° C., such as 65° C., when measured at a temperature in the range of from 25 to 300° C. and a heating rate of 2° C./min. In a particular embodiment, the endotherm is due to loss of solvent such as loss of organic solvent and/or water. In one embodiment, the organic solvent is isopropyl acetate.

In yet another embodiment, the invention relates to a crystalline form of zongertinib (form XX) characterized by having a crystal habit comprising needle-shaped crystals.

In another aspect, the invention relates to a composition comprising form XX of zongertinib as defined herein, wherein said composition is essentially free of a solid-state form of zongertinib other than form XX. For example, the invention relates to a composition comprising form XX of zongertinib as defined herein, wherein said composition comprises at most 20 wt-%, preferably at most 15 wt-%, more preferably at most 10 wt-%, most preferably at most 5, 4, 3, 2 or 1 wt-% of a solid-state form of zongertinib other than form XX, based on the weight of the composition. In one embodiment, the other solid-state form is amorphous zongertinib.

In one embodiment, the invention relates to a composition comprising form XX of zongertinib as defined herein, wherein form XX of zongertinib is present in an amount of at least 80 wt-%, preferably of at least 85 wt-%, more preferably of at least 90 wt-%, including at least 90, 91, 92, 93, 94, 95, 96, 97, 98 or 99 wt-%, and also including equal to 100 wt-%, based on the weight of the composition. The remaining material may comprise solid-state form(s) of zongertinib other than form XX. Preferably, the remaining material is amorphous zongertinib.

In another aspect, the invention relates to a process for the preparation of form XX or the composition comprising form XX of zongertinib as defined herein, said process comprising:
(i) providing a crystalline form of zongertinib (form IV) characterized by having an XRPD comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at least 15 volumes of water or a solvent mixture comprising water and isopropyl acetate at a temperature in the range of from 30 to 40° C.; and
(iii) isolating at least a part of the crystalline form XX of zongertinib or the composition comprising crystalline form XX of zongertinib obtained in (ii).

The form IV starting material in (i) can be prepared according to any one of the procedures provided in reference example 1.3.

In one embodiment, the organic solvent/water ratio in (ii) is 1:1 (vol:vol). For example, the isopropyl acetate/water ratio is 1:1 (vol:vol). In a further embodiment, the amount of solvent applied in (ii) is 15 volumes or 20 volumes. The temperature during slurrying in (ii) may be in the range of from 30 to 40° C. In a preferred embodiment, the slurrying in (ii) is performed at 35° C.

Once, the crystalline form XX of zongertinib of the present invention or the composition comprising the crystalline form XX of zongertinib of the present invention is obtained in sufficient amount, at least a part of the solid, preferably most of the solid, most preferably substantially all the solid is separated from the mother liquor in step (iii) of the above-described process. Thereby, the solid may be separated from the mother liquor by any conventional method known to the skilled person. In one embodiment, the solid is separated from the mother liquor by filtration, centrifugation, solvent evaporation and/or decantation. In a preferred embodiment, the solid is separated from the mother liquor by filtration and/or centrifugation. In a most preferred embodiment, the solid is separated from the mother liquor by filtration.

According to a further aspect, provided is a crystalline form or a composition comprising the crystalline form obtained or obtainable by:
(i) providing a crystalline form of zongertinib (form IV) characterized by having an XRPD comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at least 15 volumes of water or a solvent mixture comprising water and isopropyl acetate at a temperature in the range of from 30 to 40° C.; and
(iii) isolating at least a part of the crystalline form of zongertinib or the composition comprising crystalline form of zongertinib obtained in (ii).

In embodiments of this obtained or obtainable crystalline form or the composition comprising the crystalline form, steps (ii) and (iii) can be performed as detailed above.

The crystalline form or the composition comprising the crystalline form obtainable or obtained by the process according to this aspect and its preferred embodiments is a further object of the invention. Preferably, this crystalline form is form as defined herein and may be characterized by having an XRPD comprising the reflections as defined above for form XX. Preferably, this crystalline form corresponds to form XX as defined herein in its broadest form or in any embodiment.

In still another aspect, the invention relates to the use of form XX of zongertinib as defined herein for the preparation of a solid-state form of zongertinib or a composition comprising a solid-state form of zongertinib. In one embodiment, the prepared solid-state form of zongertinib is selected from the group consisting of form III and form IV as defined herein. In yet another embodiment, the prepared solid-state form of zongertinib is amorphous.

In one embodiment, the invention relates to the use of form XX of zongertinib as defined herein as an intermediate for the preparation of a solid-state form of zongertinib or a composition comprising a solid-state form of zongertinib. In one embodiment, the prepared solid-state form of zongertinib is selected from the group consisting of form III and form IV as defined herein. In yet another embodiment, the prepared solid-state form of zongertinib is amorphous.

In still another embodiment, the invention relates to the use of form XX of zongertinib as defined herein for the preparation of a solid-state form of zongertinib other than form XX or a composition comprising a solid-state form of zongertinib other than form XX. In one embodiment, the other solid-state form of zongertinib is selected from the group consisting of form III and form IV as defined herein. In yet another embodiment, the other solid-state form of zongertinib is amorphous.

In yet another aspect, the invention relates to a process for the preparation of a solid-state form of zongertinib or a composition comprising a solid-state form of zongertinib comprising:
(i) dissolving or suspending form XX as defined herein in a suitable solvent or in a mixture of suitable solvents; and
(ii) crystallizing or precipitating a solid-state form of zongertinib or a composition comprising a solid-state form of zongertinib from the mixture provided in (i); and
(iii) isolating the solid-state form obtained in (ii).

In one embodiment of the above defined process, the prepared solid-state form of zongertinib is selected from the group consisting of form III and form IV as defined herein. In yet another embodiment, the prepared solid-state form of zongertinib is amorphous. In still another embodiment, the prepared solid-state form of zongertinib is not form XX.

### Solid Dispersion

The crystalline zongertinib forms XXIV, XIX, XVIII and XX as described herein are useful to generate solid dispersions. Therefore, further provided herein is the use of any one of crystalline forms XXIV, XIX, XVIII and XX as described above in the broadest form or in any embodiment for the preparation of a solid dispersion comprising zongertinib and a pharmaceutically acceptable dispersion carrier. In such solid dispersion, zongertinib is preferably amorphous. In other words, the solid dispersion preferably comprises zongertinib in amorphous form.

In embodiments, the dispersion carrier is a polymer. Polymeric dispersion carriers also are denoted “dispersion polymers”. Polymers are widely used in solid dispersion formulations. Different polymeric carriers lead to solid dispersions with various properties in terms of physical stability, phase behavior and drug release rate and extent. Due to the complex nature solid dispersion formulation carrier best suited for a given API need to be tested. The dispersion polymer preferably is a neutral or acidic polymer.

In other embodiments, the dispersion carrier is a polymer that is enteric (acidic polymer) or non-enteric (neutral polymer), preferably enteric. The term “enteric polymer” refers to a pH-dependent acidic polymer that is insoluble or only slightly soluble at a low pH (e.g. at pH 1 up to but less than pH 3) but becomes soluble at a higher pH (e.g. at pH 5 and above). In certain embodiments a pH-dependent polymer may become soluble at a pH range from pH 5 and above, e.g. from pH 6 to 9, from pH 6 to 8, from pH 5 to 7, or from pH 5 to 6, which is generally less acidic than the gastric environment and roughly corresponds to pH values in the small intestine. Examples of enteric polymers include but are not limited to methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers (Eudragit® L100), shellac, cellulose acetate trimellitate, sodium alginate and zein. The term “non-enteric polymer” refers to a neutral polymer that does not show pH-dependent solubility characteristics. Examples of non-enteric polymers include but are not limited to cellulose derivatives such as methylcellulose (MC), ethylcellulose (EC), hydroxypropylcellulose (HPC), hydroxyethyl cellulose (HEC), and hydroxypropyl methylcellulose (HPMC); poly-vinyl-pyrrolidone (PVP), copovidone such as polyvinylpyrrolidone-vinyl acetate copolymer (PVP-VA); poly (ethylene glycol) PEGs; starch derivatives like cyclodextrin; Soluplus® which is an amphiphilic copolymer consisting of polyethylene glycol, polyvinyl caprolactam, and polyvinyl acetate.

In embodiments, the dispersion carrier is a polymer selected from the group consisting of hydroxypropyl methylcelluloses and esters thereof, polyvinylpyrrolidones and copolymers thereof, and polymethacrylates and copolymers thereof. The dispersion carrier may contain a mixture of two or more polymers.

In an embodiment, the hydroxypropyl methylcelluloses and esters thereof are selected from the group consisting of hydroxypropyl methyl cellulose acetate (HPMCA), hydroxypropyl methyl cellulose (HPMC), hydroxypropyl cellulose (HPC), methyl cellulose, hydroxyethyl methyl cellulose, hydroxyethyl cellulose, hydroxyethyl cellulose acetate, hydroxyethyl ethyl cellulose, hydroxypropyl methyl cellulose acetate succinate (HPMCAS), hydroxypropyl methyl cellulose phthalate (HPMCP), carboxymethyl ethyl cellulose (CMEC), cellulose acetate phthalate (CAP), cellulose acetate succinate (CAS), hydroxypropyl methyl cellulose acetate phthalate (HPMCAP), cellulose acetate trimellitate (CAT), hydroxypropyl methyl cellulose acetate trimellitate (HPMCAT), and carboxymethylcellulose acetate butyrate (CMCAB). In an embodiment, the hydroxypropyl methylcelluloses and esters thereof are selected from the group consisting of hydroxypropyl methylcellulose acetate succinate and hydroxypropyl methylcellulose, in particular hot melt extrusion-grade hydroxypropyl methylcellulose.

In an embodiment, the polyvinylpyrrolidones and copolymers thereof are selected from the group consisting of polyvinylpyrrolidone vinyl acetate copolymer (PVP-VA), polyvinyl alcohols, polyvinyl alcohol polyvinyl acetate copolymers and polyvinylpyrrolidone (PVP). Polyvinylpyrrolidone (PVP) also is commonly denoted polyvidone or povidone.

In an embodiment, polymethacrylates and copolymers thereof are selected from the group consisting of methacrylic acid-ethyl acrylate copolymer, methacrylic acid-methyl methacrylate copolymer, methyl methacrylate and methacrylic acid copolymer. Polymethacrylates and copolymers thereof are, for example, available under the brand name Eudragit® from Evonik Industries AG. Methacrylic acid-methyl methacrylate copolymer is, for example, available under the brand name Eudragit® L100.

In embodiments, the dispersion carrier is a polymer selected from the group of hydroxypropyl methylcellulose acetate succinate (HPMCAS), polyvinylpyrrolidone vinyl acetate copolymer (PVP-VA), methylacrylic acid methyl methacrylate copolymer (such as Eudragit® L100), and hot melt extrusion-grade hydroxypropyl methylcellulose (HPMC HME).

In certain embodiments, the dispersion carrier is hydroxypropyl methylcellulose acetate succinate (HPMCAS). HPMCAS also is known as hypromellose acetate succinate. Hypromellose acetate succinate (HPMCAS) can be obtained by introducing acetyl and succinoyl groups to the hydroxyl groups of the backbone of hydroxypropyl methylcellulose (HPMC) also known as hypromellose. This procedure can be carried out by known methods, for instance by treating HPMC with acetic anhydride and/or with succinic anhydride. Acetic anhydride and succinic anhydride can be reacted with hydroxypropyl methylcellulose (HPMC) under specifically controlled conditions to produce HPMCAS with varying extent of substitution of acetyl and succinoyl groups.

HPMCAS is available in several grades (L, M and H) varying in extent of substitution of acetyl and succinoyl groups, based on the content of acetyl and succinoyl groups (wt %) in the HPMCAS molecule. Any grade of HPMCAS is usable in the solid dispersion. Preferably, HPMCAS of grade L, M or H is used. In certain embodiments, the dispersion carrier is HPMCAS grade L. In certain embodiments, the dispersion carrier is HPMCAS grade M. HPMCAS grade M may comprise an acetyl content of 7-11 wt %; a succinoyl content of 10-14 wt %; methoxyl content of 21-25 wt %; and a hydroxypropoxy content of 5-9 wt %. In certain embodiments, the dispersion carrier is HPMCAS grade H. Preferably, granular HPMCAS (HPMCAS-G) is used.

In certain embodiments, the dispersion carrier is polyvinylpyrrolidone vinyl acetate copolymer (PVP-VA). Polyvinylpyrrolidone vinyl acetate copolymers are linear, random copolymers that are available by free-radical polymerization of the monomers in ratios varying from 70/30 to 30/70 vinyl acetate to vinylpyrrolidone.

In certain embodiments, the dispersion carrier is methylacrylic acid methyl methacrylate copolymer, such as Eudragit® L100. As used herein, “methylacrylic acid methyl methacrylate copolymer” is used interchangeably with “methacrylic acid methyl methacrylate copolymer”.

In certain embodiments, the dispersion carrier is a hot melt extrusion-grade hydroxypropyl methylcellulose (HPMC HME). HPMC HME refers to hydroxypropyl methylcellulose formulated via hot melt extrusion. HPMC HME is a water-soluble amorphous polymer, usually provided as a white to off-white powder, available in three grades, HPMC HME 15 LV, HPMC HME 100 LV and HPMC HME 4M, differing in regard to their molecular weight. Preferably, HPMC HME 15LV having a molecular weight (Mw) below 100 kDa is used. Further preferred, HPMC HME 100LV having a molecular weight (Mw) below 200 kDa is used.

By dispersing one or more of the crystalline forms XXIV, XIX, XVIII and XX of zongertinib, preferably on a molecular level, in a, for example polymeric, dispersion carrier, an amorphous state of zongertinib can be obtained and maintained, even when exposed to elevated temperature and/or humidity conditions, and the solid dispersion can reliably provide zongertinib in amorphous form. In embodiments of the solid dispersion, zongertinib is amorphous. The term “amorphous” as used herein refers to a condensed phase where molecules are randomly orientated and characterized by the absence of any microscopic order, with no diffraction peaks by XRPD; an amorphous solid system may be composed of a single chemical entity or may be a multi-component system containing, e.g., an API, polymer and other excipients, without stoichiometric composition. Amorphous solids generally possess crystal-like short range molecular arrangement, but no long-range order of molecular packing as found in crystalline solids. The solid-state form of a solid may be determined by X-ray powder diffraction (“XRPD”) or modulated differential scanning calorimetry (“mDSC”). In embodiments, the solid dispersion comprises amorphous zongertinib and a dispersion polymer, wherein zongertinib is substantially in amorphous solid-state form. In certain embodiments, the substantially amorphous solid-state form refers to the solid dispersion comprising at least 80 wt-% amorphous zongertinib based on a total weight of 100 wt-% of zongertinib. In certain embodiments, the substantially amorphous solid-state form refers to the solid dispersion comprising at least 85 wt-% amorphous zongertinib based on a total weight of 100 wt-% of zongertinib. In certain embodiments, the substantially amorphous solid-state form refers to the solid dispersion comprising at least 90 wt-% amorphous zongertinib based on a total weight of 100 wt-% of zongertinib. In certain embodiments, the substantially amorphous solid-state form refers to the solid dispersion comprising at least 95 wt-% amorphous zongertinib based on a total weight of 100 wt-% of zongertinib. In certain embodiments, the substantially amorphous solid-state form refers to the solid dispersion comprising at least 96, 97, 98 or 99 wt-% amorphous zongertinib based on a total weight of 100 wt-% of zongertinib. Thus, the solid dispersion can provide zongertinib in amorphous or essentially amorphous state. Such a solid dispersion can thus be referred to as an amorphous solid dispersion. In embodiments, the solid dispersion thus is an amorphous solid dispersion.

In one embodiment, the solid dispersion comprises a predetermined amount of zongertinib or a pharmaceutically acceptable salt thereof. In this context, a predetermined amount refers to the initial amount of zongertinib, or a pharmaceutically acceptable salt thereof used for the preparation of the solid dispersion.

In another embodiment, the solid dispersion comprises a therapeutically effective amount of zongertinib or a pharmaceutically acceptable salt thereof.

Solid dispersions are generally prepared by dissolving or suspending an active substance and a dispersion carrier in a solvent or mixture of solvents to form a feed solution or suspension, and then the solvent is removed from the feed solution or suspension, such as by spray-drying, to form the solid dispersion.

A further aspect of the present invention is thus a process of preparing a solid dispersion as described herein, the process comprising the steps of:
(i) providing a mixture of zongertinib and a pharmaceutically acceptable dispersion carrier and adding a solvent to obtain a solution or a suspension; and
(ii) removing the solvent from the solution or the suspension to form the solid dispersion as described herein, wherein
in step (i), zongertinib is provided as any one of crystalline forms XXIV, XIX, XVIII and/or XX or as composition comprising any one of crystalline forms XXIV, XIX, XVIII and/or XX as described above in the broadest form or in any embodiment. Preferably, in step (ii), the solid dispersion comprises zongertinib in amorphous form.

This process may further comprise the step of drying the solid dispersion obtained in step (ii).

In an embodiment, a process of preparing a solid dispersion is provided, the process comprising the steps of:
(i) providing a solution or suspension comprising zongertinib, a pharmaceutically acceptable dispersion carrier and at least one solvent; and
(ii) removing the solvent from the solution or the suspension to form the solid dispersion as described herein; and
(iii) optionally, drying the solid dispersion obtained in (ii), wherein
in step (i), the zongertinib is provided as any one of crystalline forms XXIV, XIX, XVIII and/or XX or as a composition comprising any one of crystalline forms XXIV, XIX, XVIII and/or XX as described above in the broadest form or in any embodiment.

The solution or suspension of step (i) can be referred to as a feed solution.

In embodiments, the removing of the solvent in step (ii) of the above defined processes is carried out by spray-drying, freeze drying, rotary evaporation, distillation, drum drying and/or vacuum drying. In a preferred embodiment, the removing of the solvent in step (ii) is carried out by spray-drying. Preferably, in step (ii), the solid dispersion comprises zongertinib in amorphous form.

Spray drying is generally performed by dissolving or suspending crystalline form XXIV, XIX XVIII and/or XX of zongertinib or a composition comprising any one of crystalline forms XXIV, XIX, XVIII and/or XX and the dispersion polymer in a solvent to prepare a feed solution. The feed solution may be pumped through an atomizer into a drying chamber. The feed solution can be atomized by conventional means known in the art, such as a two-fluid sonicating nozzle, a pressure nozzle, a rotating nozzle and a two-fluid non-sonicating nozzle. Then, the solvent is removed in the drying chamber to form the solid dispersion. A typical drying chamber uses hot gases, such as forced air, nitrogen, nitrogen-enriched air, or argon to dry particles. The size of the drying chamber may be adjusted to achieve particle properties or throughput.

Although the solid dispersion is preferably prepared by conventional spray drying techniques, other techniques known in the art may be used, such as melt extrusion, freeze drying, rotary evaporation, co-precipitation, KinetiSol® Dispersing Technology (KSD), drum drying, vacuum drying or other solvent removal processes.

Any solvent or mixture of solvents where the crystalline form XXIV, XIX, XVIII and/or XX or a composition comprising any one of crystalline forms XXIV, XIX, XVIII and/or XX at least partially dissolve can be used. Examples of suitable solvents that can be used individually or as mixtures include water, alcohols, such as methanol (“MeOH”), ethanol (“EtOH”), 1-propanol, 2-propanol and butanol such as n-butanol, 2-butanol, isobutanol and tert-butanol; ketones, such as acetone, methyl ethyl ketone and methyl isobutyl ketone; esters, such as methyl acetate, ethyl acetate and propyl acetate, isopropyl acetate, n-butyl acetate and isobutyl acetate; and various other solvents, such as dichloromethane (DCM), chloroform, tetrahydrofuran, acetonitrile, toluene and 1,1,1-trichloroethane. In an embodiment, the solvent referred to in any of the above-described processes and embodiments thereof is selected from the group consisting of water, alcohols, ketones, esters, dichloromethane, chloroform, tetrahydrofuran, acetonitrile, toluene, 1,1,1-trichloroethane and mixtures thereof. Mixtures of solvents with water may also be used.

In embodiments, said solvent is a mixture of dichloromethane (DCM) and methanol (MeOH). The relative amounts of DCM and MeOH in the mixture may vary. Preferably, the mixture comprises at least 25 wt-% MeOH based on a total weight of 100 wt-% of the mixture. In embodiments, the mixture comprises an excess of DCM. Still preferably, the weight:weight ratio of DCM:MeOH ranges from 25:75 to 95:5 (w/w). Preferably, DCM and MeOH are in a weight:weight ratio of 25:75, 50:50, 70:30, 75:25, 80:20, 85:15 or 90:10 (w/w). A solvent mixture of DCM:MeOH in a ratio of 90:10 (w/w) was advantageously found to enable higher throughput for spray-drying.

In embodiments, the concentration of solids in the feed solution (in particular the suspension or solution as defined in step (i) above) is in the range of from 1 to 20 wt-%, based on a total weight of 100 wt-% of the feed solution. Preferably, the concentration of solids in the feed solution is in the range of from 5 to 15 wt-%, more preferably of from 8 to 12 wt-% based on a total weight of 100 wt-% of the feed solution. For example, the concentration of solids in the feed solution is 10 wt-%, based on a total weight of 100 wt-% of the feed solution.

After removal of the solvent by spray drying, the obtained solid dispersion is optionally subjected to a drying process in order to reduce residual solvent content. In embodiments, drying is performed at a temperature in the range of from room temperature to 100° C., preferably of from 30 to 60° C., more preferably of from 35 to 45° C. For example, the drying is performed at a temperature of 40° C. In other embodiments, the drying may be performed at ambient pressure and/or under reduced pressure. For example, the drying is performed at ambient pressure or at a pressure of 900 mbar or less, more preferably of 100 mbar or less and most preferably of 50 mbar or less, such as 20 mbar or less. In still other embodiments, the drying may be performed for a period in the range of from 6 to 72 hours, preferably of from 2 to 48 hours.

Pharmaceutical compositions, such as tablets, preferably film-coated tablets, can be manufactured according to conventional methods known to a skilled person. In embodiments, the manufacturing process can comprise the steps of 1) manufacturing a solid dispersion such as by spray-drying as described herein, 2) dry granulating of the solid dispersion with one or more suitable excipient(s), 3) blending the granules with suitable disintegrant(s) and/or lubricant(s), and/or glidants 4) compressing the blend into tablet cores, and 5) optionally film-coating the tablet cores.

In embodiments of the process of preparing a solid dispersion, zongertinib in crystalline form XXIV, XIX, XVIII and/or XX or a composition comprising any one of forms XXIV, XIX, XVIII and/or XX may be provided in an amount in a range of from 5 wt-% to 95 wt-%, based on a total weight of 100 wt-% of the solid dispersion. In embodiments of the process of preparing a solid dispersion, zongertinib in crystalline form XXIV, XIX, XVIII and/or XX or a composition comprising any one of forms XXIV, XIX, XVIII and/or XX may be provided in an amount in a range of from 25 wt-% to 75 wt-%, based on a total weight of 100 wt-% of the solid dispersion. In embodiments of the process of preparing a solid dispersion, zongertinib in crystalline form XXIV, XIX, XVIII and/or XX or a composition comprising any one of forms XXIV, XIX, XVIII and/or XX may be provided in an amount in a range of from 20 wt-% to 50 wt-%, based on a total weight of 100 wt-% of the solid dispersion. In embodiments, zongertinib in crystalline form may be provided in an amount in a range of from 25 wt-% to 50 wt-%, based on a total weight of 100 wt-% of the solid dispersion. In embodiments, the dispersion carrier may be provided in an amount in a range of from 5 wt-% to 95 wt-%, based on a total weight of 100 wt-% of the solid dispersion. In embodiments, the dispersion carrier may be provided in an amount in a range of from 50 wt-% to 80 wt-%, based on a total weight of 100 wt-% of the solid dispersion. In embodiments, the dispersion carrier may be provided in an amount in a range of from 50 wt-% to 75 wt-%, based on a total weight of 100 wt-% of the solid dispersion. In embodiments, the zongertinib and the dispersion carrier may be provided in equal weight mounts. In embodiments, 50 wt-% of zongertinib and 50 wt-% of the dispersion carrier are provided. In embodiments, the weight ratio of zongertinib:the dispersion carrier in the process of preparing a solid dispersion is of 1:1 to 1:3 (w/w). In embodiments, the weight ratio of zongertinib and the dispersion carrier in the process of preparing a solid dispersion is of 1:1 (w/w).

### EXAMPLES

The following non-limiting examples are illustrative for the disclosure and are not to be construed as to be in any way limiting for the scope of the invention.

### Example 1: Preparation of Zongertinib Form XXIV, and Form XIX in Aqueous Organic Solvents

Zongertinib form IV (e.g. prepared according to any one of the procedures of reference example 1.3) was slurried in different aqueous organic solvent systems (12V, composition according to table 1) at a temperature ranging from 20 to 50° C. using a magnetic stir bar. Heat-cool-cycles were conducted using automation available on the Junior robot and as per the below program:
1. Set stirring to 350 rpm (by means of magnetic stir bars)
2. Heat to 50° C. (10° C./minute)
3. Hold at 50° C. for 150 minutes
4. Cool to 20° C. (−1° C./minute)
5. Hold at 20° C. for 120 minutes
6. Heat to 50° C. (5° C./minute)
7. Hold at 50° C. for 120 minutes
8. Cool to 20° C. (−1° C./minute)
9. Set stirring to 0 rpm
10. Hold at 20° C. for 16 hrs

The solids were collected and analyzed by XRPD. As can be seen from table 1, Form IV fully converted to Form XXIV in 1:1 (vol:vol) IPAc:water and 1:0.5 (vol:vol) THF:water, while Form XIX was obtained from 1:1 (vol:vol) EtOH:water and 1:1 (vol:vol) ACN:water, indicating the thermodynamic stability of the obtained forms in the applied aqueous organic solvent mixtures.

TABLE 1
Summary and results of form IV slurry experiments

| | Input solid | | XRPD results |
|---|---|---|---|
| Example | form | Solvent mixtures | after slurry |
| 1.1 | 1 g Form IV | 1:1 (vol:vol) IPAc:water | Form XXIV |
| 1.2 | 1 g Form IV | 1:1 (vol:vol) EtOH:water | Form XIX |
| 1.3 | 1 g Form IV | 1:1 (vol:vol) ACN:water | Form XIX |
| 1.4 | 2 g Form IV | 2:1 (vol:vol) THF:water | Form XXIV |

### Example 2: Preparation of Zongertinib Form XX in Aqueous Organic Solvents or Pure Water

### Example 2.1

A suspension of zongertinib form IV (e.g. prepared according to any one of the procedures of reference example 1.3) and zongertinib form III (e.g. prepared according to reference example 1.2) in 20V of 1:1 (vol:vol) IPAc/water was stirred using overhead agitation at 35° C. for 18 hrs. The solid was collected and analyzed by XRPD confirming receipt of form XX.

### Example 2.2

A suspension of zongertinib form IV (e.g. prepared according to any one of the procedures of reference example 1.3) and zongertinib form III (e.g. prepared according to reference example 1.2) in 20V of water was stirred using overhead agitation at 35° C. for 18 hrs. The solid was collected and analyzed by XRPD confirming receipt of form XX.

### Example 2.3

The procedures described in examples 2.1 and 2.2 also work starting from either pure form IV or pure form III.

### Example 3: Preparation of Zongertinib Form XVIII

Form XVIII is formed upon excessive drying of form XXIV and/or form XIX (e.g. prepared according to any one of examples 1.1 to 1.4 herein). Typically, drying is performed at temperatures >50° C. under vacuum.

### Example 4: X-Ray Powder Diffraction (XRPD)

XRPD was performed with a Bruker D8 Discover X-ray diffractometer. The conditions and instrument settings for the measurements are summarized in table 2. The instrument detector allows for two-dimensional diffractograms to be acquired. Solid samples were placed on a zero-background diffraction plate. The results were processed using DIFFRAC.EVA software. The diffraction pattern was integrated from two-dimensional to one-dimensional. The Kα₂ contribution was stripped, no smoothing or background correction was performed. A typical precision of the 2-Theta values is in the range of ±0.2° 2-Theta, preferably in the range of ±0.1° 2-Theta. Thus, a reflection that usually appears at 7.0 2-Theta for example can appear between 6.8° and 7.2° 2-Theta, preferably between 6.9° and 7.1° 2-Theta on most X-ray diffractometers under standard conditions.

TABLE 2
Instrumental parameters

| Parameter | Value | |
|---|---|---|
| Temperature | 20-30° | C. |
| Manufacturer | Bruker | |
| Model | D8 Discover | |
| Current | 1.0 | mA |
| Voltage | 50.0 | kV |
| Geometry | Bragg-Brentano | |
| Source type | Cu Kα, Point focus | |
| Detector type | Eiger 2R-500K (2 Dimensional) | |
| Source wavelength | 1.54056 | Å |
| Scan mode | Coupled TwoTheta/Theta, step | |
| Divergence slit | 0.1 | mm |
| Detector distance | 291.8 | mm |
| Collection time | 150 | seconds |

| | |
|---|---|
| 2Theta results range | 5.0 to 27.4 °2θ |
| Frequency of performance verification | Weekly |
| (using corundum standard) | |

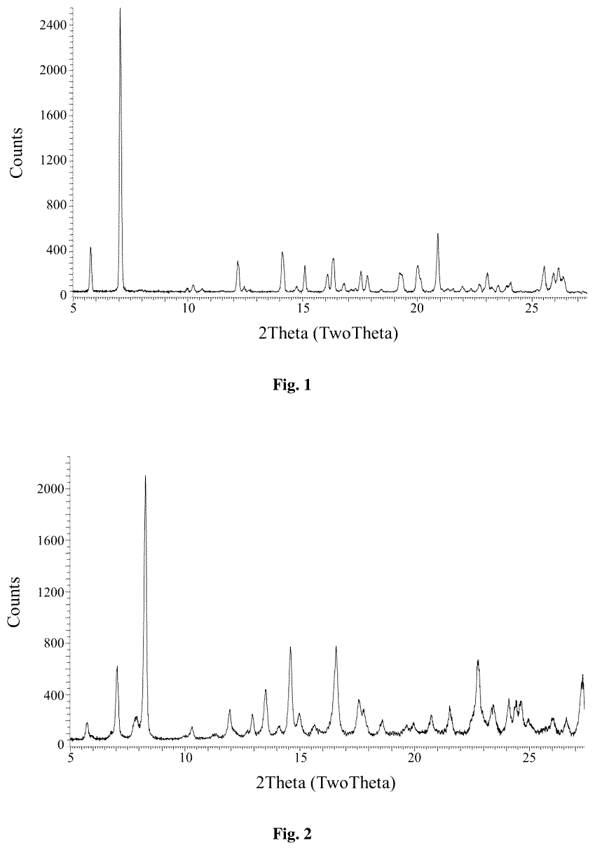

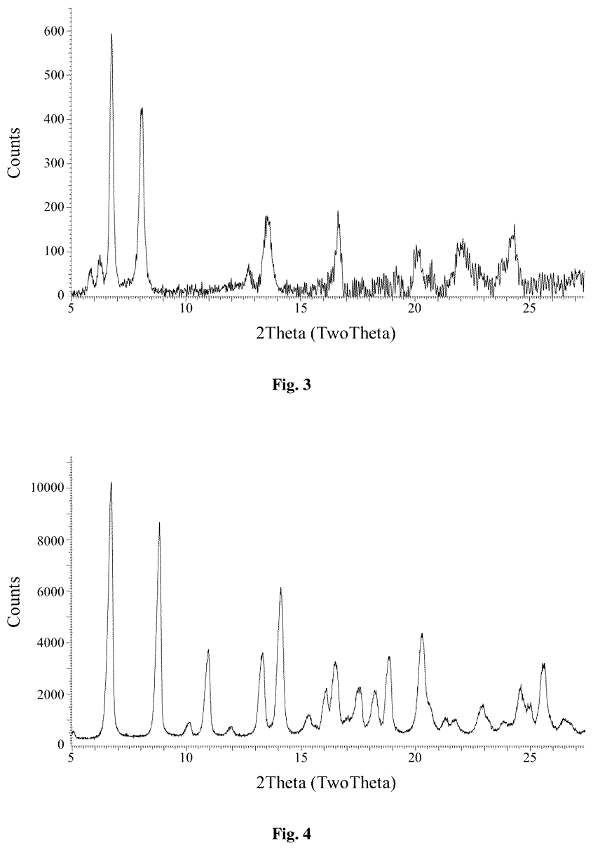

The XRPDs of each of the forms are shown in FIG. 1 (form XXIV), FIG. 2 (form XIX), FIG. 3 (form XVIII) and FIG. 4 (form XX). Each of the four forms have their own unique XRPD pattern, and each form is identifiable and characterizable by X-ray powder diffraction. For example, form XXIV shows a unique reflection at 19.3° 2-Theta which is not present in forms XIX, XVIII or XX. Form XX shows a unique reflection at 8.8° 2-Theta, which is not present in forms XXIV, XIX or XVIII. On the other hand, form XVIII shows a small unique reflection at 6.2° 2-Theta which is neither present in form XXIV, form XIX nor in form XX. Finally, Form XIX, shows a unique combination of reflections at 8.3° 2-Theta and 14.6° 2-Theta, said combination being absent in the XRPDs of forms XXIV, XVIII and XX.

### Example 5: Differential Scanning Calorimetry (DSC)

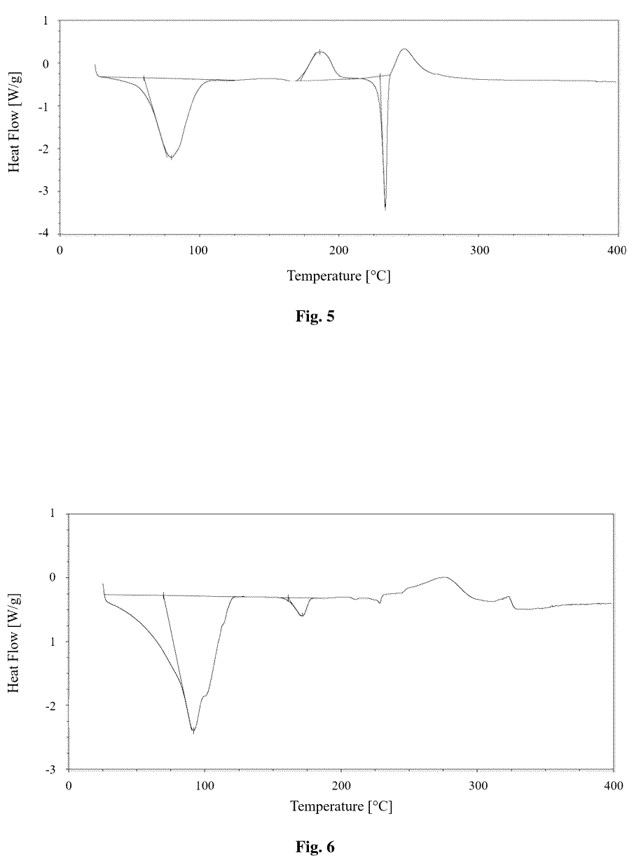

DSC was performed with a TA Instruments DSC 25 (for forms XXIV and XIX) and a Mettler Toledo DSC 3 (for form XVIII and XX), respectively. Samples (5-10 mg) were heated in aluminium pans with a sealed aluminium lid either from 25 to 400° C. at a heating rate of 10° C./min (for form XXIV and XIX) or from 25 to 300° C. at a heating rate of 5° C./min (for form XVIII) or a heating rate of 2° C./min (for form XX). Processing was completed using TRIOS software. The respective DSC curves of form XXIV, XIX, XVIII and XX are shown in FIGS. 5 to 8.

The DSC curves of form XXIV (FIG. 5) and form XIX (FIG. 6) both show a first broad endotherm, in the case of form XXIV with an onset temperature of about 60° C. and a peak temperature of about 80° C., and in the case of form XIX with an onset temperature of about 69° C. and a peak temperature of about 92° C. These endotherms are due to the release of organic solvent and water from the crystal structures of forms XXIV and XIX.

Upon further heating, the DSC curve of form XIX shows a melting endotherm with an onset temperature of about 161° C. and a peak temperature of about 171° C., which is due to melting of form XVIII, whereas the DSC curve of form XXIV shows an exotherm with an onset temperature of about 172° C. and a peak temperature of about 186° C. which reflects a crystallization event followed by a melting endotherm with an onset temperature of about 229° C. and a peak temperature of about 233° C., which corresponds to the melting of zongertinib form IV.

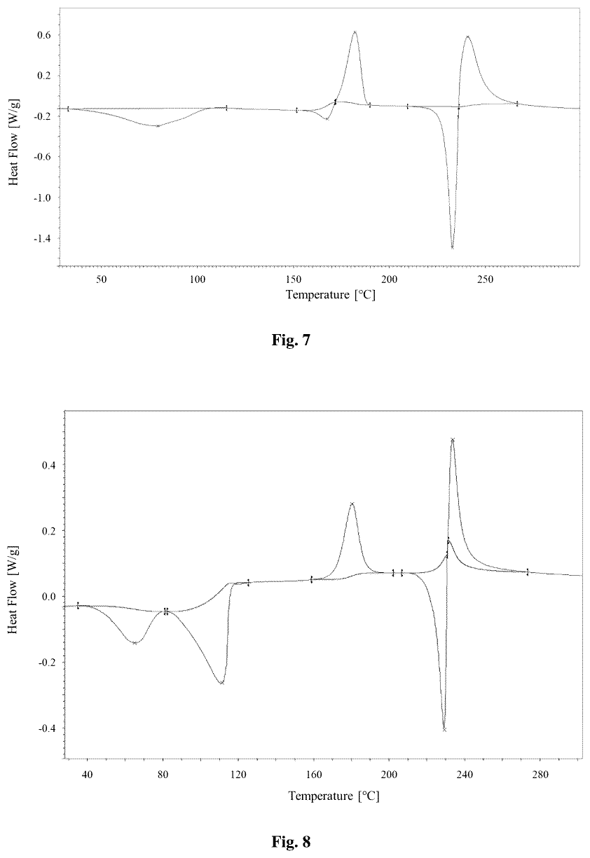

The DSC curve of form XVIII (FIG. 7) shows multiple thermal events starting with a broad endotherm having an onset temperature of about 49° C. and a peak temperature of about 80° C., which is due to dehydration. Upon further heating, the DSC curve of form XVIII shows a melting endotherm with an onset temperature of about 160° C. and a peak temperature of about 167° C. immediately followed by an exotherm indicating a recrystallization event. Finally, the recrystallized form melts as indicated by an endotherm with an onset temperature of about 228° C. and a peak temperature at about 233° C. again immediately followed by an exotherm.

The DSC curve of form XX (FIG. 8) also shows multiple thermal events starting with a first broad endotherm having an onset at a temperature of about 48° C. and a peak at a temperature of about 65° C. due to solvent release, followed by a second broad endotherm with an onset at a temperature of about 91° C. and a peak at a temperature of about 112° C. due to melting of form XX. Upon further heating, the DSC curve shows an exotherm with an onset at a temperature of about 172° C. and a peak at a temperature of about 180° C. which is due to a crystallization event. Finally, the recrystallized form melts indicated by an endotherm with an onset temperature of about 224° C. and a peak temperature at about 229° C. again immediately followed by an exotherm.

### Example 6: Thermal Gravimetric Analysis

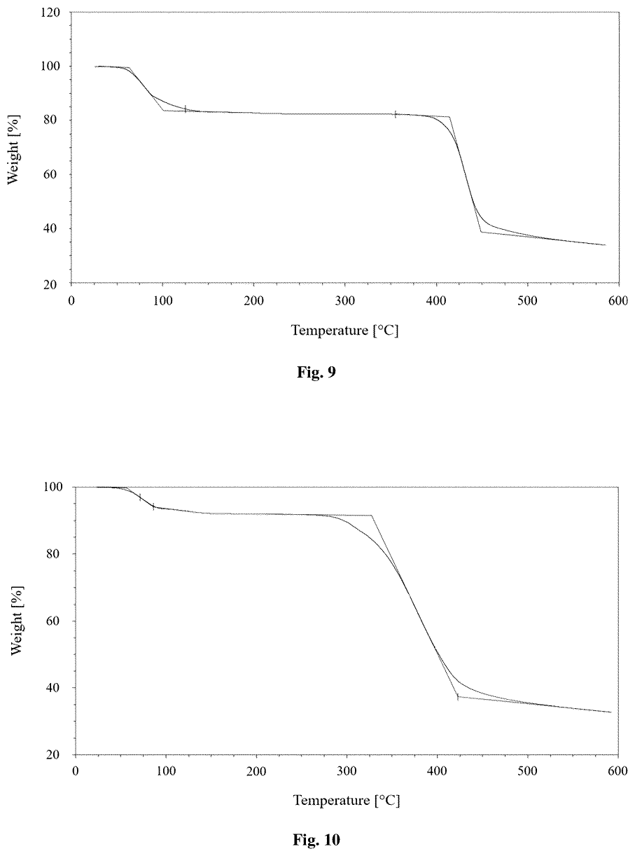

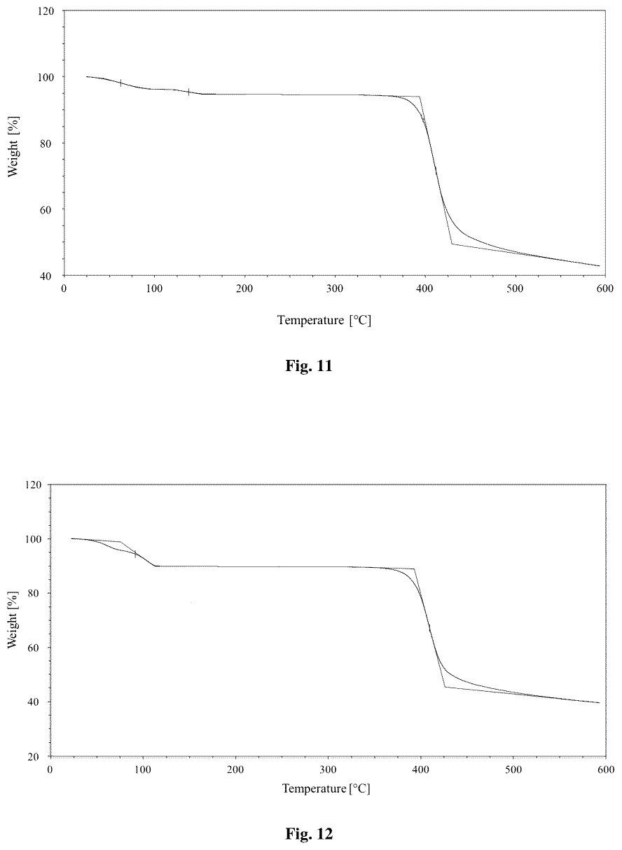

TGA was performed with a TA Instruments TGA 550. Samples (5 mg) were heated in aluminium pans with a sealed aluminium lid from ambient temperature to 600° C. at a heating rate of 20° C./min. Processing was completed using TRIOS software. The respective TGA curves of form XXIV, XIX, XVIII and XX are shown in FIGS. 9 to 12.

The TGA curve of form XXIV shows solvent evaporation starting at about 63° C. then sample decomposes starting at about 415° C.

The TGA curve of form XIX depicts solvent evaporation with an onset temperature of about 56° C. then sample decomposition starts at about 327° C.

The TGA curve of form XVIII shows two consecutive solvent evaporation events with onsets at about 80° C. and 122° C. Sample decomposition occurred at about 394° C.

The TGA curve of form XX shows two consecutive weight loss events before 100° C., which correspond to solvent evaporation then sample starts to decompose at about 393° C.

### Example 7: Polarized Light Microscopy (PLM)

Crystal habits of zongertinib forms XXIV, XIX and XX have been elucidated using polarized light microscopy. Depending on the starting material, form XVIII can be obtained in different crystal shapes. The results are summarized in table 3.

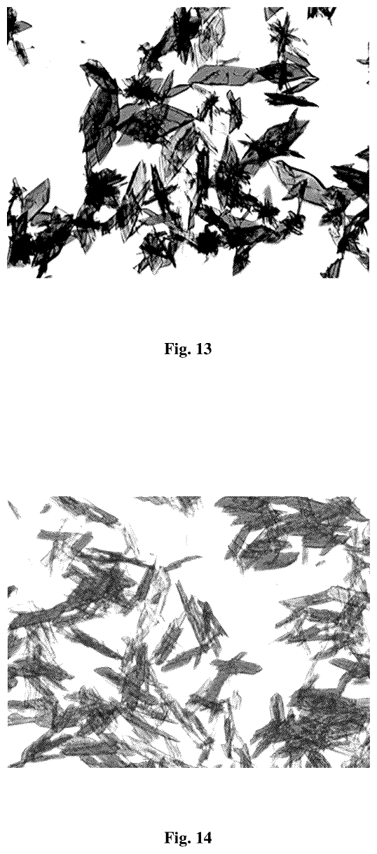

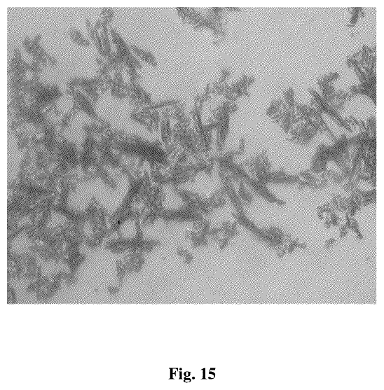

TABLE 3
PLM summary

| Form | Crystal Habit | FIG. |
|---|---|---|
| XXIV | plate-shaped and lath shaped crystals | FIG. 13 |
| XIX | lath-shaped crystals | FIG. 14 |
| XX | needle-shaped crystals | FIG. 15 |
| XVIII (drying of XXIV) | plate-shaped and lath shaped crystals | — |
| XVIII (drying of XIX) | lath-shaped crystals | — |

### Reference Example 1: Preparation of Zongertinib Staring Materials

### Reference Example 1.1—Manufacture of Zongertinib Form I

First example procedure for the preparation of form I: 19 kg of zongertinib are dissolved in a mixture of ˜54 kg THF, ˜160 kg DCM and ˜48 kg MeOH. Residual inorganic salts are removed by washing with brine (48 kg). Undissolved particulates are removed by polish filtration of the organic layer. The organic layer is then distilled to ˜160 L and the mixture is diluted with 78 kg of THF. The distillation, THF dilution, distillation sequence is repeated until levels of water and MeOH≤1.0% w/w each. Upon completion of distillations, the obtained slurry is held at ambient temperature for not longer than 12 hrs and filtered to yield form I.
Second example procedure for the preparation of form I: 6 g of zongertinib are dissolved in 75 g 5% w/w H₂O in IPA solution at 90° C. Solution is slowly cooled to 75° C. and seeded with 60 mg of form I (e.g. prepared according to the procedure described above). Mixture is agitated at 75° C. for 2 hours followed by cooling at 0.3° C./min rate to 20° C. Upon completion of cooling, solids are filtered and dried to yield form I.

### Reference Example 1.2—Manufacture of Zongertinib Form III

First example procedure for the preparation of form III: To a clean N₂-sparged vessel, form I of zongertinib (1.0 equiv. e.g. prepared according to any one of the procedures described under reference example 1.1) and IPAc (8.0 V) are charged and agitation is started. Slurry is agitated at ambient temperature for 2 hours and filtered. Solids are washed with IPAc (2.0 V) and dried in vacuum to yield form III of zongertinib.
Second example procedure for the preparation of form III: 17 kg of form I of zongertinib (e.g. prepared according to any one of the procedures described under reference example 1.1) are mixed with 271 kg of IPAc. Slurry is heated to 70° C. To the slurry, 0.2 kg of seeds of form III of zongertinib (e.g. prepared according to the first example described above) are added, and mixture is agitated for ˜16 hrs. Upon completion of hold, mixture is gradually cooled to 53° C. in ˜40 mins, then to 33° C. in ˜40 mins, then to 25° C. Obtained slurry is agitated for ˜1 hr and filtered. Solids are washed with 27 kg of IPAc and dried to yield form III.

The procedure can also be performed without addition of seeds.

### Reference Example 1.3—Manufacture of Zongertinib Form IV

First example procedure for the preparation of form IV: 300 mg zongertinib are dispersed in 3 mL of 1-BuOH. Mixture is heated to 90° C. with over-head agitation. Dissolution can be observed. Solution is cooled with the rate of 0.2° C./min to 75° C. followed by quick cooling to 20° C. Obtained slurry is held for ˜12 hrs at 20° C. while agitated and filtered to yield form IV.
Second example procedure for the preparation of form IV: zongertinib is dissolved in 10 volumes of 1-BuOH/Anisole (1:1) mixture at 110° C. Solution is subjected to distillation with slight vacuum during which most of the 1-BuOH was removed. The solution is seeded with form III seeds, held at 110° C. and a slurry is obtained. Mixture is cooled to ambient temperature while agitated and filtered to yield isolated zongertinib as form IV. This despite being seeded with form III.

1. A crystalline form of zongertinib (form XXIV) of formula (1) 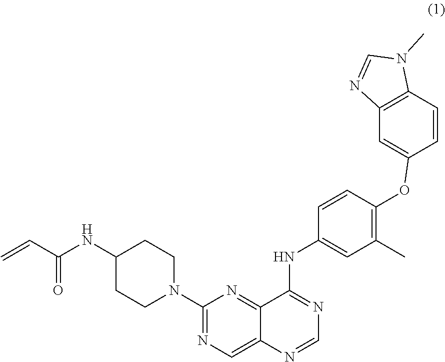 [SMILES: C=CC(=O)NC1CCN(c2ncc3ncnc(Nc4ccc(Oc5ccc6c(c5)ncn6C)c(C)c4)c3n2)CC1]
characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.2)° and (19.3±0.2)°.

2. The crystalline form of claim 1 characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):
$$( 5.8 \pm 0.2 ) ° , ( 7. \pm 0.2 ) ° , and ( 19.3 \pm 0 \text{.2} ) ° ; or$$
$$( 5.8 \pm 0.2 ) ° , ( 7. \pm 0.2 ) ° , ( 14.1 \pm 0.2 ) ° , and ( 19.3 \pm 0 \text{.2} ) ° ; or$$
$$( 5.8 \pm 0.2 ) ° , ( 7. \pm 0.2 ) ° , ( 12.2 \pm 0.2 ) ° , ( 14.1 \pm 0.2 ) ° , and ( 19.3 \pm 0 \text{.2} ) ° .$$

3. A composition comprising the crystalline form as defined in claim 1, wherein the crystalline form is present in an amount of at least 80 wt-%, based on the weight of the composition.

4. A process for the preparation of the crystalline form as defined in claim 1 comprising:
(i) providing a crystalline form of zongertinib (form IV) characterized by having an X-ray powder diffractogram comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at most 12 volumes of a solvent mixture comprising water and an organic solvent selected from isopropyl acetate or tetrahydrofuran at a temperature in the range of from 20 to 50° C.; and
(iii) isolating at least a part of the crystalline form obtained in (ii).

5. A crystalline form of zongertinib (form XIX) of formula (1) 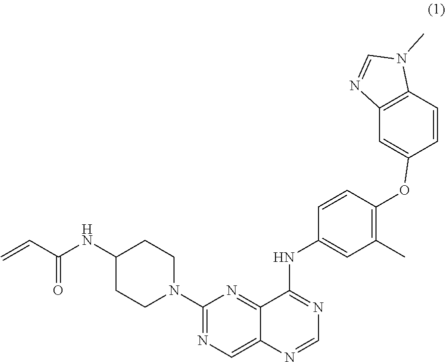 [SMILES: C=CC(=O)NC1CCN(c2ncc3ncnc(Nc4ccc(Oc5ccc6c(c5)ncn6C)c(C)c4)c3n2)CC1]
characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (8.3±0.2)° and (14.6±0.2)°.

6. The crystalline form of claim 5 characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):
$$( 7. \pm 0.2 ) ° , ( 8.3 \pm 0 \text{.2} ) ° , and ( 14.6 \pm 0 \text{.2} ) ° ; or$$
$$( 5.7 \pm 0 \text{.2} ) ° , ( 7. \pm 0.2 ) ° , ( 8.3 \pm 0 \text{.2} ) ° , and ( 14.6 \pm 0 \text{.2} ) ° ; or$$
$$( 5.7 \pm 0 \text{.2} ) ° , ( 7. \pm 0.2 ) ° , ( 8.3 \pm 0 \text{.2} ) ° , ( 14.6 \pm 0 \text{.2} ) ° , and ( 16.6 \pm 0 \text{.2} ) ° .$$

7. A composition comprising the crystalline form as defined in claim 5, wherein the crystalline form is present in an amount of at least 80 wt-%, based on the weight of the composition.

8. A process for the preparation of the crystalline form as defined in claim 5 comprising:
(i) providing a crystalline form of zongertinib (form IV) characterized by having an X-ray powder diffractogram comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at most 12 volumes of a solvent mixture comprising water and an organic solvent selected from ethanol or acetonitrile at a temperature in the range of from 20 to 50° C.; and
(iii) isolating at least a part of the crystalline form obtained in (ii).

9. A crystalline form of zongertinib (form XVIII) of formula (1) 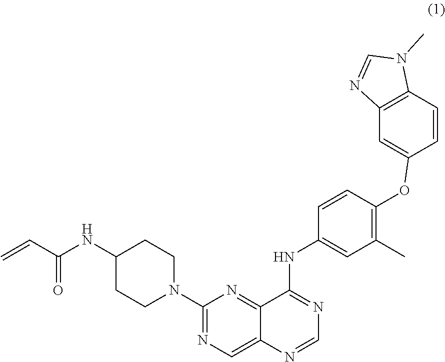 [SMILES: C=CC(=O)NC1CCN(c2ncc3ncnc(Nc4ccc(Oc5ccc6c(c5)ncn6C)c(C)c4)c3n2)CC1]
characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.2±0.2)°, (6.8±0.2)° and (8.1±0.2)°.

10. The crystalline form of claim 9 characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):
$$( 5.8 \pm 0 \text{.1} ) ° , ( 6.2 \pm 0 \text{.1} ) ° , ( 6.8 \pm 0 \text{.1} ) ° , and ( 8.1 \pm 0 \text{.1} ) ° ; or$$
$$( 5.8 \pm 0 \text{.1} ) ° , ( 6.2 \pm 0 \text{.1} ) ° , ( 6.8 \pm 0 \text{.1} ) ° , ( 8.1 \pm 0 \text{.1} ) ° , and ( 13.6 \pm 0 \text{.1} ) ° : or$$
$$( 5.8 \pm 0 \text{.1} ) ° , ( 6.2 \pm 0 \text{.1} ) ° , ( 6.8 \pm 0 \text{.1} ) ° , ( 8.1 \pm 0 \text{.1} ) ° , ( 13.6 \pm 0 \text{.1} ) ° , and ( 16.7 \pm 0 \text{.1} ) ° .$$

11. A composition comprising the crystalline form as defined in claim 9, wherein the crystalline form is present in an amount of at least 80 wt-%, based on the weight of the composition.

12. A process for the preparation of the crystalline form as defined in claim 9 comprising:
(i) providing the following crystalline form:
a) crystalline form XXIV characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.2)° and (19.3±0.2)°; or
b) crystalline form XIX characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (8.3±0.2)° and (14.6±0.2)°;
or any mixture thereof as a solid; and
(ii) at least partially removing the solvent from the crystalline form or mixture provided in (i).

13. A crystalline form of zongertinib (form XX) of formula (1) 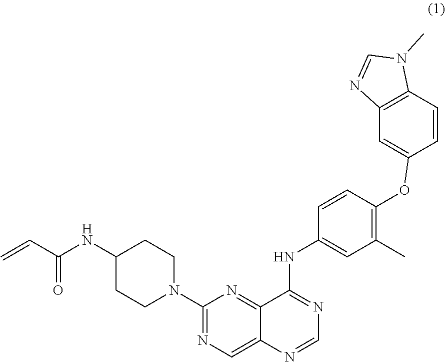 [SMILES: C=CC(=O)NC1CCN(c2ncc3ncnc(Nc4ccc(Oc5ccc6c(c5)ncn6C)c(C)c4)c3n2)CC1]
characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.7±0.2)° and (8.8±0.2)°.

14. The crystalline form of claim 13 characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}):
$$( 6.7 \pm 0 \text{.2} ) ° , ( 8.8 \pm 0 \text{.2} ) ° , and ( 10.9 \pm 0 \text{.2} ) ° ; or$$
$$( 6.7 \pm 0 \text{.2} ) ° , ( 8.8 \pm 0 \text{.2} ) ° , ( 10.9 \pm 0 \text{.2} ) ° , and ( 14.1 \pm 0.2 ) ° ; or$$
$$( 6.7 \pm 0 \text{.2} ) ° , ( 8.8 \pm 0 \text{.2} ) ° , ( 10.9 \pm 0 \text{.2} ) ° , ( 13.3 \pm 0 \text{.2} ) ° , and ( 14.1 \pm 0.2 ) ° .$$

15. A composition comprising the crystalline form as defined in claim 13, wherein the crystalline form is present in an amount of at least 80 wt-%, based on the weight of the composition.

16. A process for the preparation of the crystalline form as defined in claim 13 comprising:
(i) providing a crystalline form of zongertinib (form IV) characterized by having an X-ray powder diffractogram comprising reflections at the following 2-theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(ii) slurrying the crystalline form provided in (i) in at least 15 volumes of water or a solvent mixture comprising water and isopropyl acetate at a temperature in the range of from 30 to 40° C.; and
(iii) isolating at least a part of the crystalline form obtained in (ii).

17. A process for the preparation of a solid-state form of zongertinib or a composition comprising a solid-state form of zongertinib comprising:
(i) dissolving or suspending one or more of the following crystalline forms:
a) crystalline form XXIV characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.2)° and (19.3±0.2)°;
b) crystalline form XIX characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (8.3±0.2)° and (14.6±0.2)°;
c) crystalline form XVIII characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.2±0.2)°, (6.8±0.2)° and (8.1±0.2)°; or
d) crystalline form XX characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.7±0.2)° and (8.8±0.2)°; or compositions thereof
in a suitable solvent or in a mixture of suitable solvents; and
(ii) crystallizing or precipitating a solid-state form of zongertinib or a composition comprising a solid-state form of zongertinib from the mixture provided in (i); and
(iii) isolating the solid obtained in (ii).

18. The process of claim 17, wherein the solid-state form of zongertinib in step (iii) is selected from the group consisting of:
(i) crystalline form III characterized by having an X-ray powder diffractogram comprising reflections at the following 2-Theta angles, when measured at a temperature in the range of from 20 to 30° C. with CuKα radiation at a wavelength of 1.54056 Å (Cu—Kα₁) or 1.54184 Å (Cu—Kα_{1,2}): (6.2±0.2)°, (9.5±0.2)°, (11.4±0.2)°, (12.4±0.2)° and (16.2±0.2)°; and
(ii) crystalline form IV characterized by having an X-ray powder diffractogram comprising reflections at the following 2-Theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and
(iii) amorphous.

19. A process for preparing a solid dispersion comprising zongertinib and a pharmaceutically acceptable dispersion carrier comprising the steps of:
(i) providing a solution or suspension comprising zongertinib, a pharmaceutically acceptable dispersion carrier and at least one solvent; and
(ii) removing the solvent from the solution or the suspension to form the solid dispersion; and
(iii) optionally, drying the solid dispersion obtained in (ii), wherein in step (i), the zongertinib is provided as one or more of the crystalline forms:
a) crystalline form XXIV characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.2)° and (19.3±0.2)°;
b) crystalline form XIX characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (8.3±0.2)° and (14.6±0.2)°;
c) crystalline form XVIII characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.2±0.2)°, (6.8±0.2)° and (8.1±0.2)°; or
d) crystalline form XX characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.7±0.2)° and (8.8±0.2)°.

20. A solid-state form of zongertinib prepared by a process comprising the following steps:
(i) providing zongertinib as one or more of the following crystalline forms:
a) crystalline form XXIV characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (7.0±0.2)° and (19.3±0.2)°;
b) crystalline form XIX characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (8.3±0.2)° and (14.6±0.2)°;
c) crystalline form XVIII characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.2±0.2)°, (6.8±0.2)° and (8.1±0.2)°; or
d) crystalline form XX characterized by having an X-ray powder diffractogram comprising reflections at the following 2-Theta angles when measured at a temperature in the range of from 20 to 30° C. with Cu—Kα radiation at a wavelength of 1.54056 Å (Cu—Kα₁) or 1.54184 Å (Cu—Kα_{1,2}): (6.7±0.2)° and (8.8±0.2)°
(ii) dissolving or suspending the one or more crystalline forms of (i) in a solvent or in a mixture of solvents;
(iii) crystallizing or precipitating the solid-state form of zongertinib form the mixture provided in (ii); and
(iv) isolating the solid-state form of zongertinib obtained in (iii).

21. The solid-state form of claim 20, wherein the solid-state form of zongertinib is selected from the group consisting of: crystalline form III characterized by having an X-ray powder diffractogram comprising reflections at the following 2-Theta angles, when measured at a temperature in the range of from 20 to 30° C. with CuKα radiation at a wavelength of 1.54056 Å (Cu—Kα₁) or 1.54184 Å (Cu—Kα_{1,2}): (6.2±0.2)°, (9.5±0.2)°, (11.4±0.2)°, (12.4±0.2)° and (16.2±0.2)°; crystalline form IV characterized by having an X-ray powder diffractogram comprising reflections at the following 2-Theta angles, when measured at a temperature in the range of from 20 to 30° C. with CuKα radiation at a wavelength of 1.54056 Å (Cu—Kα₁) or 1.54184 Å (Cu—Kα_{1,2}): (5.9±0.2)°, (11.7±0.2)°, (14.7±0.2)°, (16.7±0.2)°, (18.8±0.2)° and (19.2±0.2); and amorphous.
